(19) United States
(12) Patent Application Publication
(10) Pub. No.: US 2026/0042854 A1
(43) Pub. Date: Feb. 12, 2026

(54) ANTI-GLP-1R ANTIBODIES AND USES THEREOF

(71) Applicant: SCIWIND BIOSCIENCES USA CO., LTD, San Ramon, CA (US)

(72) Inventors: Xinle WU, Beijing (CN); Haixia ZOU, Beijing (CN); Bin WANG, Beijing (CN); Rui ZHU, Beijing (CN)

(21) Appl. No.: 19/099,299

(22) PCT Filed: Aug. 1, 2023
(86) PCT No.: PCT/CN2023/110577

§ 371 Date: Jan. 28, 2025

(30) Foreign Application Priority Data
Aug. 1, 2022 (CN) 202210916213.3

(51) Int. Cl. C07K 16/28; A61K 9/00; A61K 39/00; A61P 3/10

(52) U.S. Cl. CPC C07K 16/2869; A61K 9/0019; A61P 3/10; A61K 2039/505; A61K 2039/545; C07K 2317/21; C07K 2317/52; C07K 2317/565; C07K 2317/76; C07K 2317/94

(57) ABSTRACT

Provided are related to antibodies or antigen-binding fragments thereof that specifically bind to GLP-IR, and uses thereof. The antibodies or antigen-binding fragments can antagonize the activation of GLP-IR by endogenous or exogenous GLP-1, and can be used for the diagnosis, prevention and/or treatment of GLP-IR-associated or hypoglycemia-associated diseases or disorders.

### FIELD

The present disclosure relates to antibodies, particularly anti-glucagon-like peptide 1 receptor (GLP-1R) monoclonal antibodies and fragments thereof, and the uses of the antibodies or fragments thereof for diagnosis, prevention and/or treatment of GLP-1R-associated and hypoglycemia-associated diseases.

### BACKGROUND

As obesity becomes a more and more serious problem worldwide, bariatric surgery, as one of the most effective means for weight loss, is being used by more and more obese patients. The number of patients who have undergone bariatric surgeries has increased by 50% over the past decade. Post-bariatric hypoglycemia (PBH), the most serious complication associated with bariatric surgery, is estimated to affect 10-30% of patients. PBH generally occurs 1-3 hours after a meal, and it will become more severe with the increase in carbohydrate intake, which will lead to dizziness, epilepsy, confusion and even death of the patients, seriously affecting their self-care ability. For patients who have undergone Roux-en-Y gastric bypass, the concentration of glucagon-like peptide 1 (GLP-1) in postprandial blood can be as high as more than 10 times that of normal people, and can reach more than 50 times in hypoglycemic patients. Elevated level of GLP-1 stimulates the excessive secretion of insulin, leading to hypoglycemia. To date, there is no effective method for the treatment of PBH. Eiger Biopharma's Avexitide (exendin 9-39), a GLP-1R antagonist as a polypeptide, has clinically demonstrated the efficacy of reversing hypoglycemia. The polypeptide is currently in phase III clinical research. Due to its very short half-life (˜30 min), patients need to inject Avexitide subcutaneously once or twice a day. Such high-frequency of administration may lead to poor compliance of patients with chronic diseases.

At present, Avexitide, as the only GLP-1R antagonist in clinical research, having the disadvantages such as short half-life and high frequency of administration, fails to meet the diverse therapeutic needs of the patients with GLP-1R-associated diseases. It is urgent to develop a long-acting GLP-1R antagonist, so as to greatly reduce the frequency of administration and improve the compliance and experience of the patients.

### SUMMARY OF THE DISCLOSURE

The present disclosure provides a novel monoclonal antibody or antigen-binding fragment thereof, a coding nucleic acid, a vector, a cell and a composition. The monoclonal antibody or antigen-binding fragment thereof of the present disclosure has the characteristics of high specificity, long half-life, remarkable drug efficacy in vivo and in vitro, and the like. At the same time, the present disclosure provides a method for treating and/or preventing GLP-1R-associated diseases, and use of the manufacture of a medicament for treating and/or preventing GLP-1R-associated diseases.

In one aspect, the present disclosure provides a monoclonal antibody or antigen-binding fragment thereof, wherein said monoclonal antibody or antigen-binding fragment thereof comprises heavy chain complementary determining region 1 (HCDR1), HCDR2 and HCDR3 of amino acid sequences of SEQ ID NO: 2, SEQ ID NO: 3 and SEQ ID NO: 4, respectively; and light chain complementary determining region 1 (LCDR1), LCDR2 and LCDR3 of amino acid sequences of SEQ ID NO: 6, SEQ ID NO: 7 and SEQ ID NO: 8, respectively; preferably, said monoclonal antibody or antigen-binding fragment thereof comprises HCVR of the amino acid sequence of SEQ ID NO: 1, and LCVR of the amino acid sequence of SEQ ID NO: 5; more preferably, said monoclonal antibody or antigen-binding fragment thereof comprises a heavy chain of the amino acid sequence of SEQ ID NO: 9, and a light chain of the amino acid sequence of SEQ ID NO: 10.

In one aspect, the present disclosure provides a monoclonal antibody or antigen-binding fragment thereof, wherein said monoclonal antibody or antigen-binding fragment thereof comprises HCDR1, HCDR2 and HCDR3 of amino acid sequences of SEQ ID NO: 2, SEQ ID NO: 3 and SEQ ID NO: 22, respectively; and LCDR1, LCDR2 and LCDR3 of amino acid sequences of SEQ ID NO: 6, SEQ ID NO: 7 and SEQ ID NO: 24, respectively; preferably, said monoclonal antibody or antigen-binding fragment thereof comprises HCVR of the amino acid sequence of SEQ ID NO: 21, and LCVR of the amino acid sequence of SEQ ID NO: 23; more preferably, said monoclonal antibody or antigen-binding fragment thereof comprises a heavy chain of the amino acid sequence of SEQ ID NO: 25 or SEQ ID NO: 27, and a light chain of the amino acid sequence of SEQ ID NO: 26.

In one aspect, the present disclosure provides a monoclonal antibody or antigen-binding fragment thereof that specifically binds to the glucagon-like peptide 1 receptor (GLP-1R). The monoclonal antibody or antigen-binding fragment thereof comprises an amino acid sequence that has one or more conservative amino acid substitutions in HCDRs in total, as compared to the amino acid sequences of the HCDRs as defined by SEQ ID NO: 2, SEQ ID NO: 3 and SEQ ID NO: 4; preferably, the number of said conservative amino acid substitutions is 1, 2 or 3; preferably, said conservative amino acid substitution(s) is (only) located in HCDR3; more preferably, the number of said conservative amino acid substitution is 1 and said conservative amino acid substitution is located in HCDR3. The monoclonal antibody or antigen-binding fragment thereof comprises an amino acid sequence that has one or more conservative amino acid substitutions in LCDRs in total, as compared to the amino acid sequences of the LCDRs as defined by SEQ ID NO: 6, SEQ ID NO: 7 and SEQ ID NO: 8; preferably, the number of said conservative amino acid substitutions is 1, 2 or 3; preferably, the said conservative amino acid substitution(s) is (only) located in LCDR3; more preferably, the number of said conservative amino acid substitution is 1 and said conservative amino acid substitution is located in LCDR3.

In some embodiments, the monoclonal antibody or antigen-binding fragment thereof comprises: an HCVR of the amino acid sequence of SEQ ID NO: 1 or 21, or having at least 90% (e.g., at least 91%, 92%, 93%, 94%, 95%, 96%, 97%, 98% or 99%) identity to the amino acid sequence of SEQ ID NO: 1 or 21; and/or an LCVR of the amino acid sequence of SEQ ID NO: 5 or 23, or having at least 90% (e.g., at least 91%, 92%, 93%, 94%, 95%, 96%, 97%, 98% or 99%) identity to the amino acid sequence of SEQ ID NO: 5 or 23. In a preferred embodiment, the monoclonal antibody or antigen-binding fragment thereof contains an HCVR of the amino acid sequence of SEQ ID NO:1 and an LCVR of the amino acid sequence of SEQ ID NO:5. In another preferred embodiment, the monoclonal antibody or antigen-binding fragment thereof contains an HCVR of the amino acid sequence of SEQ ID NO:21 and an LCVR of the amino acid sequence of SEQ ID NO:23.

In some embodiments, the monoclonal antibody or antigen-binding fragment thereof is a fully human antibody.

In some embodiments, the monoclonal antibody or antigen-binding fragment thereof is a full-length IgG antibody; preferably, the monoclonal antibody or antigen-binding fragment thereof comprises a human antibody constant region; more preferably, the monoclonal antibody or antigen-binding fragment thereof comprises a human antibody variable region and a human antibody constant region.

In some embodiments, the heavy chain of the monoclonal antibody or antigen-binding fragment thereof comprises a constant region selected from that of human IgG1, IgG2, IgG3, IgG4 or conventional variants thereof; preferably, the light chains of the monoclonal antibody or antigen-binding fragment thereof comprises a constant region selected from that of human K chain, A chain or conventional variants thereof.

In some embodiments, the monoclonal antibody or antigen-binding fragment thereof has specificity to human GLP-1R: preferably, the monoclonal antibody or antigen-binding fragment thereof is capable of antagonizing the activation of human GLP-1R by GLP-1; more preferably, the monoclonal antibody or antigen-binding fragment thereof does not have specificity to mouse GLP-1R.

In some embodiments, the monoclonal antibody or antigen-binding fragment thereof is an antagonist of GLP-1R and is capable of antagonizing the activation of human GLP-1R by GLP-1. The experiment or test for the binding of an antibody of this disclosure refers to the experiment to verify the interaction of an antibody with a specific antigen molecule or the influence or activity of such interaction on a particular effect under different treatment conditions. The experiment includes but is not limited to that verifying the binding capability or activity of an antibody to a particular form of antigen, that verifying the antagonistic ability or activity of an antibody against the binding of a specific antigen to other molecules, etc. For the above purposes of verification, any feasible verification methods which are known to those skilled in the art can be taken, including but not limited to enzyme-linked immunosorbent assay (ELISA), flow cytometry assay, surface plasmon resonance assay and cell reporter gene assay, etc.

In some embodiments, the monoclonal antibody or antigen-binding fragment thereof has one or more properties selected from the group consisting of:
(1) binding to extracellular domain of hGLP-1R (NP_002053.3, aa 24-145) with an EC₅₀ of less than or equal to 70 pM, as measured by ELISA; preferably, binding to extracellular domain of hGLP-1R (NP_002053.3, aa 24-145) with an EC₅₀ of less than or equal to 50 pM; further preferably, binding to extracellular domain of hGLP-1R (NP_002053.3, aa 24-145) with an EC₅₀ of less than or equal to 20 pM;
(2) binding to hGLP-1R on the cell membrane with an EC₅₀ of less than or equal to 50 nM, as measured by flow cytometry: preferably, binding to hGLP-1R on the cell membrane with an EC₅₀ of less than or equal to 30 nM; further preferably, binding to hGLP-1R on the cell membrane with an EC₅₀ of less than or equal to 15 nM; and
(3) antagonizing the activity of exogenous GLP-1 of 2.5 ng/ml with an IC₅₀ of less than or equal to 100 nM, as measured by hGLP-1R-CRE-Luciferase-HEK293 reporter gene assay; preferably, antagonizes the activity of exogenous GLP-1 of 2.5 ng/ml with an IC₅₀ of less than or equal to 50 nM; further preferably, antagonizes the activity of exogenous GLP-1 of 2.5 ng/mL with an IC₅₀ of less than or equal to 20 nM.

In some embodiments, the monoclonal antibody or antigen-binding fragment thereof has a long half-life in vivo. For example, the plasma half-life of the monoclonal antibody or antigen-binding fragment thereof is greater than 150 hours after a single subcutaneous injection to a humanized GLP-1R mouse, preferably greater than 200 hours.

In one aspect, the present disclosure provides a monoclonal antibody or antigen-binding fragment thereof, which competes for binding to GLP-1R with any one of the aforementioned monoclonal antibodies or antigen-binding fragments thereof.

In one aspect, the present disclosure also provides a nucleic acid encoding any one of the aforementioned monoclonal antibodies or antigen-binding fragments thereof. In one embodiment, the monoclonal antibody or antigen-binding fragment thereof encoded by the nucleic acid molecule comprises HCDR1 of the amino acid sequence of SEQ ID NO: 2, HCDR2 of the amino acid sequence of SEQ ID NO: 3, and HCDR3 of the amino acid sequence of SEQ ID NO: 4; and/or LCDR1 of the amino acid sequence of SEQ ID NO: 6, LCDR2 of the amino acid sequence of SEQ ID NO: 7, and LCDR3 of the amino acid sequence of SEQ ID NO: 8. In another embodiment, the monoclonal antibody or antigen-binding fragment thereof encoded by the nucleic acid molecule comprises HCDR1 of the amino acid sequence of SEQ ID NO: 2, HCDR2 of the amino acid sequence of SEQ ID NO: 3, and HCDR3 of the amino acid sequence of SEQ ID NO: 22; and/or LCDR 1 of the amino acid sequence of SEQ ID NO: 6, LCDR2 of the amino acid sequence of SEQ ID NO: 7, and LCDR3 of the amino acid sequence of SEQ ID NO: 24.

The nucleic acids can be a polynucleotide which includes coding sequence, or a polynucleotide which includes additional coding and/or non-coding sequence. The nucleic acids described in the present disclosure can be DNA or RNA. DNA includes, without limitation, cDNA, genomic DNA, and synthetic DNA. DNA can be single-stranded or double-stranded. DNA can be either the coding strand or the non-coding strand. The nucleic acids encoding the monoclonal antibodies or antigen-binding fragments thereof described in the present disclosure include, but are not limited to, coding sequences that only encode the mature monoclonal antibodies or antigen-binding fragments thereof, coding sequences that encode mature monoclonal antibodies or antigen-binding fragments thereof and other additional coding sequences, and coding sequences (and optional additional coding sequences) that encode mature monoclonal antibody or antigen-binding fragments thereof and non-coding sequences.

The present disclosure also relates to nucleic acids which hybridize to the above-mentioned sequences and have at least 50%, preferably at least 70%, more preferably at least 80% identity between the two sequences. In particular, the present disclosure relates to nucleic acids that hybridize under strict conditions to the nucleic acids of the present disclosure. In the present disclosure, “strict conditions” refer to the conditions in which: (1) hybridization and elution are carried out at low ionic strength and high temperature, such as 0.2×SSC, 0.1% SDS, 60° C.; or (2) a denaturant is added during hybridization, such as 50% (v/v) formamide, 0.1% calf serum/0.1% Ficoll, 42° C., etc.; or (3) hybridization occurs only when the identity between the two sequences is at least 90% or more, more preferably 95% or more. Also, the polypeptides encoded by the hybridizable nucleic acids have the same biological function and activity as the mature polypeptides.

The full-length nucleic acid sequence of the antibody of the present disclosure, or the fragment of the nucleic acid sequence, can usually be obtained by PCR amplification, recombination or chemical synthesis. Once a relevant sequence has been obtained, recombination methods can be used to obtain the relevant sequences in bulk. This is usually done by cloning the sequence into a vector, transferring it into a cell, and isolating it from the propagated host cell by conventional methods. Biomolecules (nucleic acids, monoclonal antibodies and antigen-binding fragments thereof, multivalent monoclonal antibodies, etc.) described in the present disclosure include biomolecules in an isolated form.

In one aspect, the present disclosure also provides a vector comprising a nucleic acid encoding any one of the above-mentioned monoclonal antibodies or antigen-binding fragments thereof. The vector of the present disclosure includes, but is not limited to, a viral vector, such as an adenovirus vector, a retrovirus vector, or an adeno-associated virus vector; and a non-viral vector, such as a plasmid and a transposon vector. Preferably, the vector is an expression vector, which is preferably a plasmid vector, further preferably a eukaryotic expression vector, more preferably a CHO cell expression vector, such as pcDNA3.4.

In one aspect, the present disclosure also provides cells for expressing any one of the aforementioned monoclonal antibodies or antigen-binding fragments thereof which contain any one of the aforementioned monoclonal antibodies or antigen-binding fragments thereof, or the nucleic acids or vectors which encode any one of the aforementioned monoclonal antibodies or an antigen-binding fragment thereof. Preferably, the cell is a host cell comprising the above-mentioned expression vector or nucleic acid. In one aspect of the present disclosure, the host cell comprises but is not limited to a mammalian cell, an insect cell, a plant cell, a fungal cell, and a prokaryotic cell. Representative examples include Escherichia coli and Streptomyces; a bacterial cell of Salmonella typhimurium; a fungal cell such as yeast; an insect cell of Drosophila S2 or Sf9; and an animal cell such as CHO, COS7, and 293 cells. Preferably, the host cell provided by the present disclosure for expressing the antibody or the antigen-binding fragment thereof that binds to GLP-1R is a CHO cell.

Transformation of host cells with DNA can be carried out by conventional techniques well known to a person skilled in the art. When the host is a prokaryote such as Escherichia coli, competent cells that can absorb DNA can be harvested after the exponential growth phase and treated with a CaCl₂) method. The steps used are well known in the art. Another method is to use MgCl₂. If necessary, transformation can also be carried out by electroporation. When the host is a eukaryote, transfection methods can be used, including a calcium phosphate co-precipitation method, a conventional mechanical method such as microinjection and electroporation, and liposome packaging.

In the process of expressing the proteins encoded by the nucleic acids of the present disclosure, conventional culture methods can be used. Depending on the host cell used, the culture medium used for cultivation can be selected from various conventional culture mediums. The culture is performed under conditions suitable for host cell growth. When host cells are grown to an appropriate density, the cells may be cultured for an additional period by inducing the selected promoter using an appropriate method (e.g., temperature changes or chemical induction).

The monoclonal antibodies or antigen-binding fragments thereof in the above-mentioned method can be expressed inside the cells or on the cell membranes, or secreted outside the cells. If necessary, the physical, chemical, and other characteristics can be used to separate and purify the monoclonal antibody or antigen-binding fragment thereof, and the like by various separation methods. These methods are well known to a person skilled in the art. Examples of these methods include but are not limited to: conventional renaturation treatment, treatment by a protein precipitant (salt precipitation), centrifugation, lysis by osmosis, sonication, super centrifugation, molecular sieve chromatography (gel filtration), adsorption chromatography, ion exchange chromatography, high performance liquid chromatography (HPLC), and any other liquid chromatography, and a combination thereof.

In one aspect, the present disclosure also provides a pharmaceutical composition, which comprises the monoclonal antibody or antigen-binding fragment thereof, nucleic acid, vector or cell aforementioned in any aspect of the present disclosure. Preferably, the pharmaceutical composition further comprises a pharmaceutically acceptable excipient. More preferably, the pharmaceutically acceptable excipients include one or more of the following: a pharmaceutically acceptable solvent, a dispersant, an additive, a plasticizer, etc. Generally, the monoclonal antibodies or antigen-binding fragments thereof of the present disclosure can be formulated in non-toxic, inert and pharmaceutically acceptable carrier mediums. The formulated pharmaceutical composition can be administrated in conventional routes including but not limited to subcutaneous, intraperitoneal, intravenous, intramuscular or topical administration.

In one aspect, the present disclosure also provides a kit comprising any one of the above-mentioned monoclonal antibodies or antigen-binding fragments thereof, nucleic acids and/or cells.

In some embodiments, the kit further comprises a testing reagent, a negative control, and a positive control for testing the GLP-1R antigen-antibody reaction.

The monoclonal antibody or antigen-binding fragment thereof according to the present disclosure has wide biological and clinical application. The application relates to various fields such as diagnosis or treatment of GLP-1R-associated diseases, basic medical research and biological research. A preferred application is for clinical diagnosis or treatment of GLP-1R-associated diseases.

In one aspect, the present disclosure provides a method of treating, preventing or ameliorating at least one symptom or condition of a GLP-1R-associated disease or disorder, wherein said method comprises administering a therapeutically effective amount of the monoclonal antibody or antigen-binding fragment thereof of the present disclosure to a subject. In some embodiments, the GLP-1R-associated disease or disorder is a GLP-1R-hyperactivity-associated disease, preferably hypoglycemia, most preferably post-bariatric hypoglycemia. In some embodiments, a pharmaceutical composition of the present disclosure is administered in combination with a second therapeutic agent.

In one aspect, the present disclosure also provides a method of treating, preventing or ameliorating at least one symptom or condition of a hypoglycemia-associated disease or disorder, wherein said method comprises administering a therapeutically effective amount of the monoclonal antibody or antigen-binding fragment thereof of the present disclosure to a subject. In some embodiments, the hypoglycemia-associated disease or disorder is preferably neurological damage or developmental complications caused by hypoglycemia or congenital hyperinsulinism (HI) (said HI includes but is not limited to the types of perinatal stress-induced transient HI, single-gene defect induced monogenic HI, and syndrome-associated HI such as Beckwith-Wiedemann syndrome). In some embodiments, the symptom or condition is preferably hypoglycemia, cerebral damage, developmental delay, feeding disorder, learning disability, and/or epilepsy. In some embodiments, a pharmaceutical composition of the present disclosure is administered in combination with a second therapeutic agent.

In one aspect, the present disclosure provides uses of any of the aforementioned monoclonal antibodies or antigen-binding fragments thereof, nucleic acids, vectors, or cells in the manufacture of a medicament for the prevention, treatment or amelioration of GLP-1R-associated or hypoglycemia-associated diseases or disorders and/or in the preparation of a kit for diagnosing or testing the associated diseases or disorders.

According to one aspect of the present disclosure, the monoclonal antibody or antigen-binding fragment thereof provided by the present disclosure has one or more of the following advantages: it antagonizes GLP-1R and inhibits the activation of GLP-1R by GLP-1, particularly, such effect is specific to human GLP-1R; it has a long in vivo half-life; it antagonizes GLP-1 significantly, and so on.

### BRIEF DESCRIPTION OF THE DRAWINGS

FIG. 1A shows the relationship between the absorbance at 450 nm and the log concentration (nM) of the antibodies in an ELISA to measure the binding capability of P16288 or P16289 antibody to the extracellular domain of hGLP-1R.

FIG. 1B shows the relationship between the average fluorescence intensity and the log concentration (nM) of the antibodies in a flow cytometric assay to measure the binding capability of P16288, P16289 or IgG1 (P02183, as negative control) antibody to the full-length hGLP-1R on the cell membrane.

FIG. 2 shows the relationship between the inhibitory rate of the activation of human GLP-1R by GLP-1 and the log concentration (nM) of the P16288 or P16289 antibody.

FIG. 3A shows the relationship between the inhibitory rate of the activation of human GLP-1R by GLP-1 and the log concentration (μM) of the P16288 or P16289 antibody.

FIG. 3B shows the relationship between the inhibitory rate of the activation of monkey GLP-1R by GLP-1 and the log concentration (μM) of the P16288 or P16289 antibody.

FIG. 3C shows the relationship between the inhibitory rate of the activation of mouse GLP-1R by GLP-1 and the log concentration (μM) of the P16288 or P16289 antibody or Avexitide.

FIG. 4A shows the change of blood glucose concentration from 0 to 180 minutes in different groups after GLP-1+glucose treatment on Day 1 of intraperitoneal glucose tolerance test (IPGTT) in humanized GLP-1R mouse model.

FIG. 4B shows the area under the curve (AUC) of blood glucose concentration from 0 to 180 minutes in different groups after GLP-1+glucose treatment on Day 1 of IPGTT in humanized GLP-1R mouse model.

FIG. 4C shows the change of blood glucose concentration from 0 to 180 minutes in different groups after GLP-1+glucose treatment on Day 8 of IPGTT in humanized GLP-1R mouse model.

FIG. 4D shows the AUC of blood glucose concentration from 0 to 180 minutes in different groups after GLP-1+glucose treatment on Day 8 of IPGTT in humanized GLP-1R mouse model.

FIG. 4E shows the change of blood glucose concentration from 0 to 180 minutes in different groups after GLP-1+glucose treatment on Day 15 of IPGTT in humanized GLP-1R mouse model.

FIG. 4F shows the AUC of blood glucose concentration from 0 to 180 minutes in different groups after GLP-1+glucose treatment on Day 15 of IPGTT in humanized GLP-1R mouse model.

FIG. 5A shows the change of blood glucose concentration from 0 to 240 minutes in different groups after oral administration of glucose solution on Day 1 of oral glucose tolerance test (OGTT) in humanized GLP-1R mouse model.

FIG. 5B shows the AUC of blood glucose concentration from 0 to 240 minutes in different groups after oral administration of glucose solution on Day 1 of OGTT in humanized GLP-1R mouse model.

FIG. 5C shows the change of blood glucose concentration from 0 to 180 minutes in different groups after oral administration of glucose solution on Day 8 of OGTT in humanized GLP-1R mouse model.

FIG. 5D shows the AUC of blood glucose concentration from 0 to 180 minutes in different groups after oral administration of glucose solution on Day 8 of OGTT in humanized GLP-1R mouse model.

FIG. 6A shows the change of blood glucose concentration from 0 to 180 minutes in different groups after GLP-1+glucose treatment of IPGTT in C57BL/6 mouse model.

FIG. 6B shows the AUC of blood glucose concentration from 0 to 180 minutes in different groups after GLP-1+glucose treatment of IPGTT in C57BL/6 mouse model.

FIG. 7A shows the change of blood glucose concentration from 0 to 270 minutes in different groups after oral administration of glucose solution of OGTT in C57BL/6 mouse model.

FIG. 7B shows the AUC of blood glucose concentration from 0 to 270 minutes in different groups after oral administration of glucose solution of OGTT in C57BL/6 mouse model.

FIG. 8 shows the pharmacokinetics of the anti-GLP-1R antibody P16288 or P16289 after single subcutaneous administration in SD rats.

FIG. 9 shows the relationship between the inhibitory rate of the activation of human GLP-1R by GLP-1 and the log concentration (μM) of the P16288 or P16888 antibody or Avexitide.

FIG. 10 shows the relationship between the inhibitory rate of the activation of monkey GLP-1R by GLP-1 and the log concentration (μM) of the P16288 or P16888 antibody or Avexitide.

FIG. 11A shows the change of blood glucose concentration from 0 to 180 minutes in different groups after GLP-1+glucose treatment on Day 1 of IPGTT in humanized GLP-1R mouse model.

FIG. 11B shows the AUC of blood glucose concentration from 0 to 180 minutes in different groups after GLP-1+glucose treatment on Day 1 of IPGTT in humanized GLP-1R mouse model.

FIG. 12A shows the change of blood glucose concentration from 0 to 180 minutes in different groups after GLP-1+glucose treatment on Day 1 of IPGTT in humanized GLP-1R mouse model.

FIG. 12B shows the change of blood glucose concentration from 0 to 180 minutes in different groups after GLP-1+glucose treatment on Day 8 of IPGTT in humanized GLP-1R mouse model.

FIG. 12C shows the change of blood glucose concentration from 0 to 180 minutes in different groups after GLP-1+glucose treatment on Day 15 of IPGTT in humanized GLP-1R mouse model.

FIG. 12D shows the change of blood glucose concentration from 0 to 180 minutes in different groups after GLP-1+glucose treatment on Day 22 of IPGTT in humanized GLP-1R mouse model.

FIG. 13 shows the pharmacokinetics of the P16888 antibody in humanized GLP-1R mouse model.

FIG. 14 shows the pharmacokinetics of the P16888 and P16891 antibodies in cynomolgus monkey model.

### DETAILED DESCRIPTION

The present disclosure can be better understood with reference to the following examples. However, it is to be understood that the following examples are for the purpose of describing particular embodiments only, and not to be understood as limiting the scope of the present disclosure in any way.

The term “GLP-1R” refers to the glucagon-like peptide 1 receptor. Glucagon-like peptide 1 (GLP-1) is a 31-amino-acid peptide hormone released from intestinal L cells following nutrient consumption. The binding of GLP-1 to GLP-1R potentiates glucose-induced secretion of insulin from pancreatic beta cells, increases insulin expression, inhibits beta-cell apoptosis, promotes beta-cell neogenesis, reduces glucagon secretion, delays gastric emptying, promotes satiety and increases peripheral glucose disposal.

The term “antibody”, as used herein, is intended to refer to an immunoglobulin molecule comprised of four polypeptide chains, two heavy (H) chains and two light (L) chains inter-connected by disulfide bonds (i.e., “full antibody molecules”), as well as a multimer thereof (e.g., IgM) and/or antigen-binding fragment thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3. CDR3, FR4. In certain embodiments, the FRs of the antibody (or antigen binding fragment thereof) may be identical to the human germline sequences or may be naturally or artificially modified. An amino acid consensus sequence may be defined based on a side-by-side analysis of two or more CDRs.

The term “antigen-binding fragment” of an antibody, as used herein, include any naturally occurring, enzymatically obtainable, synthetic, or genetically engineered polypeptide, protein or glycoprotein that specifically binds to an antigen to form a complex. The term “antigen-binding fragment” of an antibody as used herein, refers to one or more fragments of an antibody that retain the ability to bind to GLP-1R.

The term “fully human antibody”, as used herein, is not intended to include mAbs in which CDR sequences derived from the germline of another mammalian species (e.g., mouse), have been grafted onto human FR sequences. The term includes antibodies that are recombinantly produced in a non-human mammal, or in cells of a non-human mammal. The term is not intended to include antibodies isolated from or generated in a human subject.

The term “conservative amino acid substitution”, as used herein, refers to one substitution in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the similarity percentage or degree may be adjusted upwards to correct for the conservative nature of the substitution. Means for making this adjustment are well known to those of skill in the art. See, e.g., Pearson (1994) Methods Mol. Biol. 24:307-331. Examples of groups of amino acids that have side chains with similar chemical properties include: 1) aliphatic side chains: glycine, alanine, valine, leucine and isoleucine: 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative substitution is any change having a positive value in the PAM250 log-likelihood matrix disclosed in Gonnet et al., (1992) Science 256:1443-45. A “moderately conservative” substitution is any change having a nonnegative value in the PAM250 log-likelihood matrix.

### Glossary

IC₅₀: Half-maximal inhibitory concentration.
EC₅₀: Half-maximal effective concentration.
T_{1/2}: Elimination half-life.
Tₘₐₓ: Time to peak drug concentration.
Cₘₐₓ: Peak concentration.
AUC: Area under the concentration-time curve.
AUC_{0-inf obs}: AUC extrapolated to infinity, based on the last observed concentration (obs).
MRT_{0-inf obs}: Mean residence time extrapolated to infinity, based on the last observed concentration (obs).

### EXAMPLE 1: PREPARATION OF ANTI-GLP-1R ANTIBODIES

Human antibodies from the naive human Fab antibody phage library constructed by Sanyou Biopharmaceuticals (Shanghai) Co., Ltd. were cross-screened by the recombinant protein of the extracellular domain of hGLP-1R (NP_002053.3, 24-145aa) in solid phase and liquid phase and by hGLP-1R-HEK293 cell (GenScript Biotech Corporation), using immuno tubes and Kingfisher magnetic bead purification system (a magnetic bead screener, Thermo Fisher Scientific). Fabs with binding specificity towards the extracellular domain of the antigen and the cell were enriched by elution with trypsin and glycine-hydrochloric acid, respectively.

The enriched output sets were tested by ELISA and FACS, and further, monoclones were selected for verification of binding properties. Finally, full-length antibodies containing the Fab with antigen binding capability were constructed by linking the Fabs which resulted from the screen to the constant regions of IgG4 (S228P). Nucleic acid sequences encoding the full-length antibodies were introduced into pcDNA3.4 (Biointron), a eukaryotic expression vector. By using CHO cell expression system, full-length antibodies were obtained after transfection, incubation, and purification by Protein A affinity chromatography. The result of the verification, as illustrated in Example 2, shows that both P16288 and P16289 have high ability of binding to the extracellular domain of hGLP-1R. However, as shown in the result of the FACS assay which tests the antibody for its capability of binding to the receptor on the cell membrane, only P16288 bound strongly to hGLP-1R on the cell membrane, whereas P16289 bound weakly.

Mutations of D92E in LCDR and D97E in HCDR of the sequence of P16288 were introduced to obtain the new antibody P16888. A YTE mutation was further introduced into the Fc region of P16888 to obtain P16891. Thereafter, based on the CHO cell expression system, full-length antibodies were obtained after transfection, incubation, and purification by Protein A affinity chromatography.

Table 1 shows the amino acid sequences of the light chains, heavy chains and CDRs of the anti-GLP-1R antibodies P16288, P16289, P16888 and P16891.

TABLE 1

| Antibody | Light Chain | Heavy Chain |
|---|---|---|
| P16288 | DIQLTQSPSFLSASVGDRVTITCRASQGISS | QVQLVQSGAEMKKPGASVKVSCKTSGYRFSTYVISWVR |
| | YLAWYQQKPGRAPKLLIYSASTLQRGVPS | QTPGQGLEWMGWISPYTGDIDYAQKFQGRVTITADESTS |
| | RFSGSGSGTDFTLTISSLQPEDYATYYCQQA | TAYMELSRLRSDDTAVYYCARERDGYNFPGMDVWGQGT |
| | DSFPPSFGPGTKVDIKRTVAAPSVFIFPPSDE | TVTVSSASTKGPSVFPLAPCSRSTSESTAALGCLVKDYFPE |
| | QLKSGTASVVCLLNNFYPREAKVQWKVD | PVTVSWNSGALTSGVHTFPAVLQSSGLYSLSSVVTVPSSSL |
| | NALQSGNSQESVTEQDSKDSTYSLSSTLTL | GTKTYTCNVDHKPSNTKVDKRVESKYGPPCPPCPAPEFL |
| | SKADYEKHKVYACEVTHQGLSSPVTKSFN | GGPSVFLFPPKPKDTLMISRTPEVTCVVVDVSQEDPEVQF |
| | RGEC (SEQ ID NO: 10) | NWYVDGVEVHNAKTKPREEQFNSTYRVVSVLTVLHQD |
| | LCDR1: RASQGISSYLA (SEQ ID NO: 6) | WLNGKEYKCKVSNKGLPSSIEKTISKAKGQPREPQVYTL |
| | LCDR2: SASTLQR (SEQ ID NO: 7) | PPSQEEMTKNQVSLTCLVKGFYPSDIAVEWESNGQPENN |
| | LCDR3: QQADSFPPS (SEQ ID NO: 8) | YKTTPPVLDSDGSFFLYSRLTVDKSRWQEGNVFSCSVMH |
| | | EALHNHYTQKSLSLSLGK (SEQ ID NO: 9) |
| | | HCDR1: TYVIS (SEQ ID NO: 2) |
| | | HCDR2: WISPYTGDIDYAQKFQG (SEQ ID NO: 3) |
| | | HCDR3: ERDGYNFPGMDV (SEQ ID NO: 4) |
| P16289 | DIQLTQSPSSLSASVGDRVTITCRASQGVSR | EVQLVETGGGVVQPGRSLRLSCAASGFTFSSYAMHWVR |
| | ALAWYQQKPGKAPKLLIYDASYLEDGVPS | QAPGKGLEWVAVISYDGSNKYYADSVKGRFTISRDNSK |
| | RFSGSGSGTDFTLTISSLQPEDFATYYCQQS | NTLYLQMNSLRAEDTAVYYCAREGYSSSWTYYYYYYM |
| | YSTPYTFGQGTKLEIKRTVAAPSVFIFPPSD | DVWGKGTMVTVSSASTKGPSVFPLAPCSRSTSESTAALG |
| | EQLKSGTASVVCLLNNFYPREAKVQWKV | CLVKDYFPEPVTVSWNSGALTSGVHTFPAVLQSSGLYSL |
| | DNALQSGNSQESVTEQDSKDSTYSLSSTLT | SSVVTVPSSSLGTKTYTCNVDHKPSNTKVDKRVESKYGP |
| | LSKADYEKHKVYACEVTHQGLSSPVTKSF | PCPPCPAPEFLGGPSVFLFPPKPKDTLMISRTPEVTCVVVD |
| | NRGEC (SEQ ID NO: 20) | VSQEDPEVQFNWYVDGVEVHNAKTKPREEQFNSTYRVV |
| | LCDR1: RASQGVSRALA (SEQ ID NO: 16) | SVLTVLHQDWLNGKEYKCKVSNKGLPSSIEKTISKAKGQ |
| | LCDR2: DASYLED (SEQ ID NO: 17) | PREPQVYTLPPSQEEMTKNQVSLTCLVKGFYPSDIAVEW |
| | LCDR3: QQSYSTPYT (SEQ ID NO: 18) | ESNGQPENNYKTTPPVLDSDGSFFLYSRLTVDKSRWQEG |
| | | NVFSCSVMHEALHNHYTQKSLSLSLGK (SEQ ID NO: 19) |
| | | HCDR1: GFTFSSYAMH (SEQ ID NO: 12) |
| | | HCDR2: VISYDGSNKYYADSVKG (SEQ ID NO: 13) |
| | | HCDR3: AREGYSSSWTYYYYYYMDV (SEQ ID NO: 14) |
| P16888 | DIQLTQSPSFLSASVGDRVTITCRASQGISS | QVQLVQSGAEMKKPGASVKVSCKTSGYRFSTYVISWVR |
| | YLAWYQQKPGRAPKLLIYSASTLQRGVPS | QTPGQGLEWMGWISPYTGDIDYAQKFQGRVTITADESTS |
| | RFSGSGSGTDFTLTISSLQPEDYATYYCQQA | TAYMELSRLRSDDTAVYYCAREREGYNFPGMDVWGQGT |
| | ESFPPSFGPGTKVDIKRTVAAPSVFIFPPSDE | TVTVSSASTKGPSVFPLAPCSRSTSESTAALGCLVKDYFPE |
| | QLKSGTASVVCLLNNFYPREAKVQWKVD | PVTVSWNSGALTSGVHTFPAVLQSSGLYSLSSVVTVPSSSL |
| | NALQSGNSQESVTEQDSKDSTYSLSSTLTL | GTKTYTCNVDHKPSNTKVDKRVESKYGPPCPPCPAPEFL |
| | SKADYEKHKVYACEVTHQGLSSPVTKSFN | GGPSVFLFPPKPKDTLMISRTPEVTCVVVDVSQEDPEVQF |
| | RGEC (SEQ ID NO: 26) | NWYVDGVEVHNAKTKPREEQFNSTYRVVSVLTVLHQD |
| | LCDR1: RASQGISSYLA (SEQ ID NO: 6) | WLNGKEYKCKVSNKGLPSSIEKTISKAKGQPREPQVYTL |
| | LCDR2: SASTLQR (SEQ ID NO: 7) | PPSQEEMTKNQVSLTCLVKGFYPSDIAVEWESNGQPENN |
| | LCDR3: QQAESFPPS (SEQ ID NO: 24) | YKTTPPVLDSDGSFFLYSRLTVDKSRWQEGNVFSCSVMH |
| | | EALHNHYTQKSLSLSLGK (SEQ ID NO: 25) |
| | | HCDR1: TYVIS (SEQ ID NO: 2) |
| | | HCDR2: WISPYTGDIDYAQKFQG (SEQ ID NO: 3) |
| | | HCDR3: EREGYNFPGMDV (SEQ ID NO: 22) |
| P16891 | DIQLTQSPSFLSASVGDRVTITCRASQGISS | QVQLVQSGAEMKKPGASVKVSCKTSGYRFSTYVISWVR |
| | YLAWYQQKPGRAPKLLIYSASTLQRGVPS | QTPGQGLEWMGWISPYTGDIDYAQKFQGRVTITADESTS |
| | RFSGSGSGTDFTLTISSLQPEDYATYYCQQA | TAYMELSRLRSDDTAVYYCAREREGYNFPGMDVWGQGT |
| | ESFPPSFGPGTKVDIKRTVAAPSVFIFPPSDE | TVTVSSASTKGPSVFPLAPCSRSTSESTAALGCLVKDYFPE |
| | QLKSGTASVVCLLNNFYPREAKVQWKVD | PVTVSWNSGALTSGVHTFPAVLQSSGLYSLSSVVTVPSSSL |
| | NALQSGNSQESVTEQDSKDSTYSLSSTLTL | GTKTYTCNVDHKPSNTKVDKRVESKYGPPCPPCPAPEFL |
| | SKADYEKHKVYACEVTHQGLSSPVTKSFN | GGPSVFLFPPKPKDTLYITREPEVTCVVVDVSQEDPEVQF |
| | RGEC (SEQ ID NO: 26) | NWYVDGVEVHNAKTKPREEQFNSTYRVVSVLTVLHQD |
| | LCDR1: RASQGISSYLA (SEQ ID NO: 6) | WLNGKEYKCKVSNKGLPSSIEKTISKAKGQPREPQVYTL |
| | LCDR2: SASTLQR (SEQ ID NO: 7) | PPSQEEMTKNQVSLTCLVKGFYPSDIAVEWESNGQPENN |
| | LCDR3: QQAESFPPS (SEQ ID NO: 24) | YKTTPPVLDSDGSFFLYSRLTVDKSRWQEGNVFSCSVMH |
| | | EALHNHYTQKSLSLSLGK (SEQ ID NO: 27) |
| | | HCDR1: TYVIS (SEQ ID NO: 2) |
| | | HCDR2: WISPYTGDIDYAQKFQG (SEQ ID NO: 3) |
| | | HCDR3: EREGYNFPGMDV (SEQ ID NO: 22) |

### EXAMPLE 2: BINDING CAPABILITIES OF THE ANTIBODIES TO THE ANTIGEN

### 2.1. An ELISA Experiment to Measure the Binding Capabilities of the Antibodies to the Antigen

The extracellular domain of hGLP-1R (NP_002053.3, aa 24-145) was expressed as a recombinant protein. An ELISA plate was coated with 10 μg/mL of hGLP-1R recombinant protein in PBS and incubated overnight at 4° C. The coating solution was removed thereafter and the plate was washed with PBST (PBS+0.05% Tween). Next, the plate was blocked with 1% BSA, washed with PBST, then added with 50 μL antibody (P16288 or P16289) of 6.7, 2.2, 0.44, 0.089, 0.018, 0.0036, 0.00071 or 0 nM, and incubated at room temperature for 60 minutes. After washed with PBST, the plate was added with horseradish peroxidase-labeled anti-human Fc antibody and incubated for 60 minutes. Then, after washed with PBST, the plate was added with TMB substrate for coloration, which was finally terminated by adding 0.5 M H₂SO₄. The absorbance was measured with microplate reader at 450 nm. The data were analyzed by four-parameter curve fitting with GraphPad Prism 9, and EC₅₀ was calculated.

TABLE 2

| | |
|---|---|
| P16288 | EC₅₀ = 13.0 pM |
| P16289 | EC₅₀ = 4.8 pM |

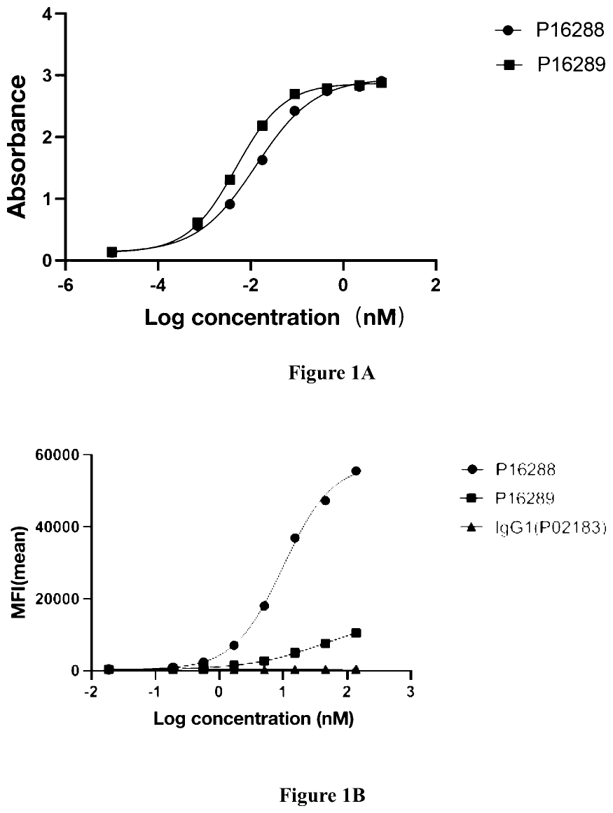

As shown in FIG. 1A and Table 2, both P16288 and P16289 antibodies have binding capabilities to the extracellular domain of hGLP-1R.

### 2.2. A Flow Cytometry Assay to Measure the Binding Capabilities of the Antibodies to the Antigen

HEK293 cells overexpressing hGLP-1R (GenScript Biotech Corporation) were washed with FACS buffer, then added with 50 μL antibody (P16288, P16289, or IgG1 (Sanyou Biopharmaceuticals (Shanghai) Co., Ltd.) as negative control) of 137.0, 45.7, 15.2, 5.1, 1.69, 0.56, 0.19 or 0.018 nM and incubated at 4° C. for 60 minutes. After centrifugation and washing, the cells were added with PE-labeled anti-human IgG Fc (Abcam, ab98596), and incubated at 4° C. for 30 minutes. After centrifugation and washing, the cells were re-suspended into a FACS buffer. The mean fluorescence intensities (MFI) were measured with flow cytometry. The data were analyzed by four-parameter curve fitting with GraphPad Prism 9, and EC₅₀ was calculated.

TABLE 3

| | |
|---|---|
| P16288 | EC₅₀ = 9.9 nM |
| P16289 | EC₅₀ > 137 nM |

As shown in FIG. 1B and Table 3, P16288 binds strongly to hGLP-1R on the cell membrane, whereas P16289 has a low binding capability to the receptor.

Therefore, P16288 but not P16289 has the ability to bind to both the extracellular domain of hGLP-1R and the hGLP-1R protein on the cell membrane.

### EXAMPLE 3: A CELL-BASED ASSAY ON THE ANTAGONISM OF THE ANTIBODIES TO hGLP-1R

The effect of the antibodies and dosage thereof on the activity of native GLP-1 (7-37aa, R&D Systems, cat no. 5374/1) were investigated in an hGLP-1R-CRE-Luciferase-HEK293 reporter gene assay.

HEK293 cells overexpressing hGLP-1R were transfected with a plasmid containing a luciferase expression cassette driven by multiple copies of cAMP response elements (CRE), to obtain a stably transfected hGLP-1R-CRE-Luciferase-HEK293 cell line containing the luciferase-expressing plasmid.

50 μL of hGLP-1R-CRE-Luciferase-HEK293 (6*10⁵ cells/mL) was added to a 96-well plate, followed by 50 μL P16288 of ten levels of concentrations in a 3-fold dilution gradient of 5240 nM, 1747 nM, 582 nM and so on, P16289 of ten levels of concentrations in a 3-fold dilution gradient of 2825 nM, 942 nM, 313 nM and so on, or equal volume of culture medium (minimum and maximum inhibition control groups). The plate was incubated at 37° C. in a 5% CO₂ incubator for 30 minutes, then added with GLP-1 to the minimal inhibition control group and antibody treatment groups to reach a final concentration of 2.5 ng/mL, and culture medium of equal volume to the maximal inhibition group, and further incubated overnight. 100 μL of Bright-Glo reagent (Promega Corporation) was added, and the plate was tapped gently to facilitate the mixing of the solution. Chemiluminescence value was read with microplate reader after three minutes. The maximum inhibition control group without GLP-1 treatment was defined as 100% inhibition rate, and the minimum inhibition control group treated with 2.5 ng/mL GLP-1 alone was defined as 0% inhibition rate. The data were analyzed by four-parameter curve fitting with GraphPad Prism 9, and IC₅₀ was calculated.

TABLE 4

| | |
|---|---|
| P16288 | IC₅₀ = 13.4 nM |
| P16289 | IC₅₀ > 2825 nM |

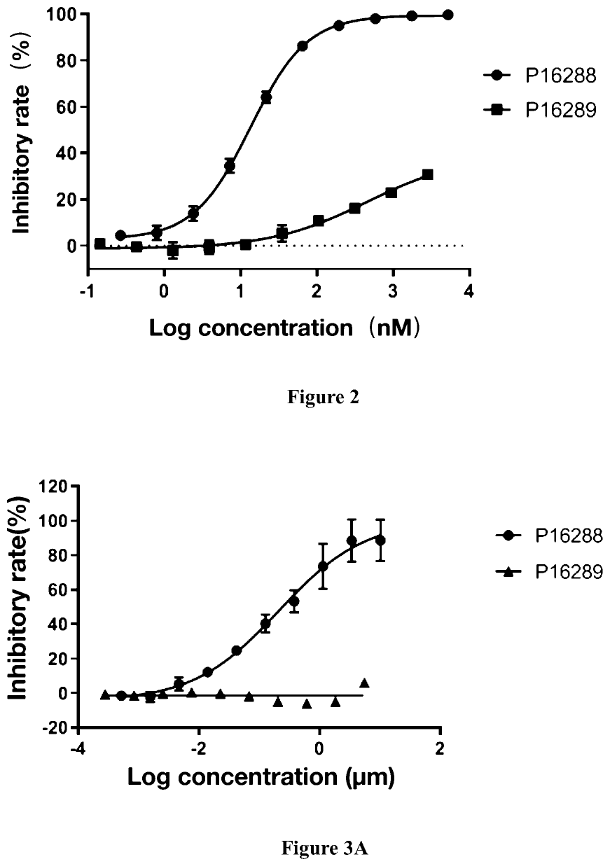

In the experiments in which GLP-1 was incubated with the cells alone, GLP-1 could activate the expression of hGLP-1R-mediated reporter gene in a dose-dependent manner. As shown in FIG. 2 and Table 4, the activity of native GLP-1 is antagonized by P16288 antibody in a dose-dependent manner, while P16289 antibody, which binds very weakly to hGLP-1R on the cell membrane, cannot antagonize the activity of native GLP-1 effectively.

### EXAMPLE 4: A STUDY ON THE ANTAGONISM OF THE ANTIBODIES TO GLP-1R OF DIFFERENT SPECIES

HEK293 cells (ATCC, cat no. CRL-1573) were transfected with GLP-1R_pcDNA3.1/G418 (+) plasmids which can transcribe the mRNA of human (NM_002062.5), monkey (JN033215.1) and mouse (NM_021332.2) GLP-1R polypeptide. 48 hours after the transfection, 50 μL P16288 of ten levels of concentrations in a 3-fold dilution gradient of 10.2 μM, 3.4 μM, 1.13 μM and so on, P16289 of ten levels of concentrations in a 3-fold dilution gradient of 5.5 μM, 1.83 μM, 0.61 μM and so on, or equal volume of culture medium (minimum and maximum inhibition control groups) were added. The plate was incubated at 37° C. for 30 minutes, then added with 1 ng/ml (for human or monkey receptor) or 6 ng/ml (for mouse receptor) of GLP-1 to the minimal inhibition control group and antibody treatment groups, and culture medium of equal volume to the maximal inhibition group. The plate was further incubated at 37° C. for 120 minutes. Finally, the CAMP level was measured with cAMP HTRF kit (Cisbio, 62AM4PEB). The maximum inhibition control group without GLP-1 treatment was defined as 100% inhibition rate, and the minimum inhibition control group treated with GLP-1 alone was defined as 0% inhibition rate. The data were analyzed by four-parameter curve fitting with GraphPad Prism 9, and IC₅₀ was calculated.

TABLE 5

| | IC₅₀ (nM) | | |
|---|---|---|---|
| Sample | Human GLP-1R | Monkey GLP-1R | Mouse GLP-1R |
| P16288 | 211.2 | 162.2 | >10200 |
| P16289 | >5500 | >5500 | >5500 |

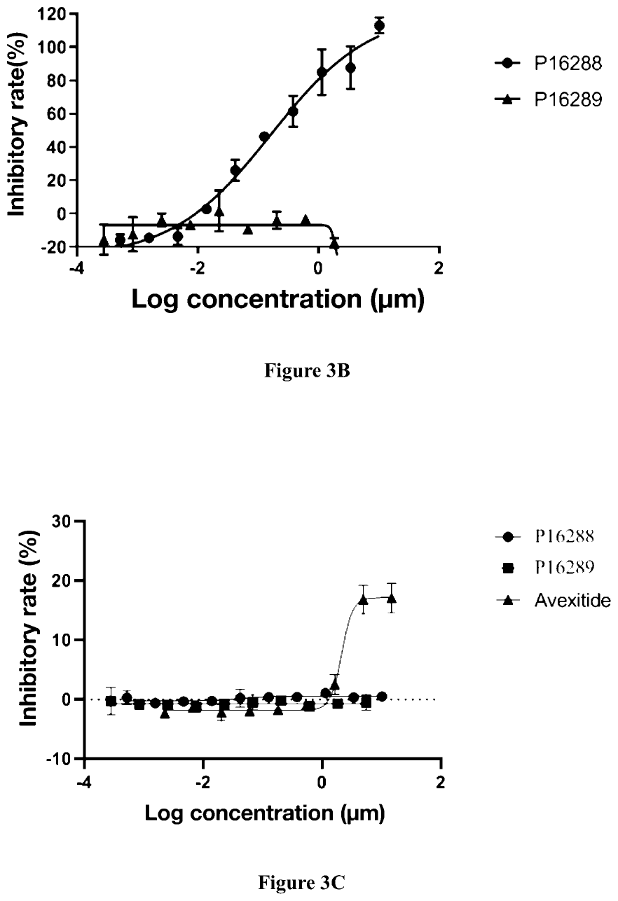

The results illustrated in FIGS. 3A-C and Table 5 show that, as for the activation of GLP-1R by GLP-1. P16288 can antagonize the activation of human and monkey GLP-1R by GLP-1 at a similar level, but cannot antagonize the activation of mouse GLP-1R. The effect of Avexitide on the activation of mouse GLP-1R by GLP-1 in GLP-1R-expressing HEK293 cells was investigated in the same protocol. The results show that Avexitide does antagonize the activation of mouse GLP-1R by GLP-1.

### EXAMPLE 5: A STUDY OF ANTIBODIES IN INTRAPERITONEAL GLUCOSE TOLERANCE TEST (IPGTT) IN HUMANIZED GLP-1R MICE (EXOGENOUS GLP-1 ADDED)

10-week-old female humanized GLP-1R mice (25-28 g, Shanghai Model Organisms Center Inc.) were assigned into 5 groups, with 5 mice in each group, and each group was treated according to the scheme in Table 6. The antibody treatment groups were subcutaneously injected with antibody, while the other groups were subcutaneously injected with vehicle (PBS) once a week for 2 consecutive weeks. IPGTTs were performed one day after the administration. The first subcutaneous administration of the antibody was on Day 0, and the second subcutaneous administration was on Day 7. After 18-hour fasting (i.e., Day 1, Day 8 and Day 15; mice were also fasted on Day 14), exogenous GLP-1 (7-37, R&D System, cat no. 5374/1) and glucose solution were dosed by intraperitoneal injection at 25.0 nmol/kg and 13.9 mmol/kg, respectively.

TABLE 6

| | Dose of | Dose of | Dose of |
|---|---|---|---|
| Group | Antibody | GLP-1 | Glucose |
| Vehicle (control) | none | none | 13.9 mmol/kg |
| 25 nmol/kg GLP-1 | none | 25.0 nmol/kg | 13.9 mmol/kg |
| 200 nmol/kg P16288 + | 200 nmol/kg | 25.0 nmol/kg | 13.9 mmol/kg |
| GLP-1 | P16288 | | |
| 100 nmol/kg P16288 + | 100 nmol/kg | 25.0 nmol/kg | 13.9 mmol/kg |
| GLP-1 | P16288 | | |
| 200 nmol/kg P16289 + | 200 nmol/kg | 25.0 nmol/kg | 13.9 mmol/kg |
| GLP-1 | P16289 | | |

### Methods of Blood Glucose Measurement and of Statistics:

The blood glucose levels of the mice before (0 min) and 15 min, 30 min, 60 min, 90 min, 120 min and 180 min after the administration of glucose were measured with blood glucose meter. The AUCs of the blood glucose-time curves of each mouse from 0 to 180 min were calculated with GraphPad Prism 9. The blood glucose levels and the AUCs were shown in mean±standard error of mean (SEM) manner and the differences among groups were compared. Statistical differences were analyzed with One-way ANOVA. As compared with the group treated with GLP-1 alone, * stands for p<0.05; ** stands for p<0.01; *** stands for p<0.001; **** stands for p<0.0001; ns stands for no significance. The significance markers were not shown in the line chart since they would overlap and be difficult to distinguish.

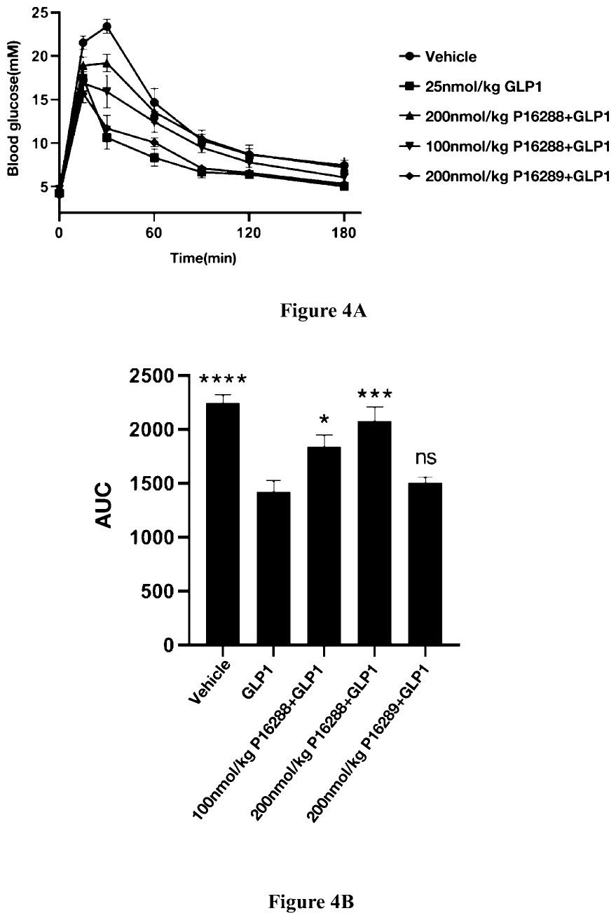

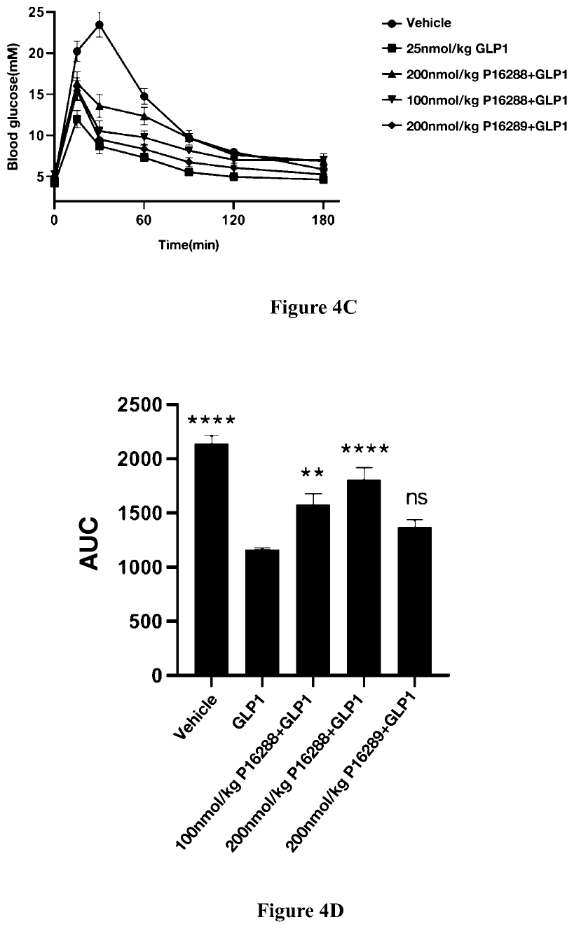

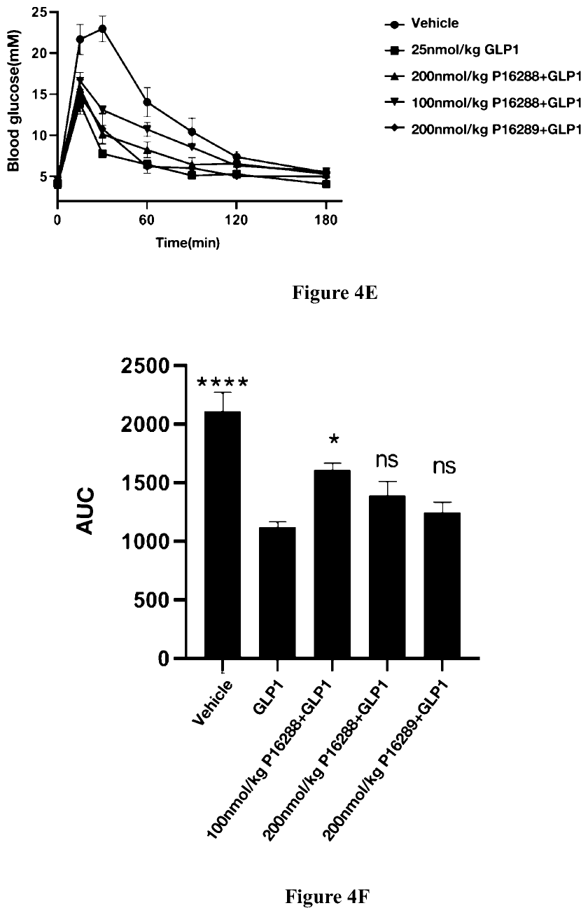

As shown in FIG. 4A-F, the addition of GLP-1 (25 nmol/kg) can effectively lower the blood glucose levels in humanized GLP-1R mice. P16288 antibody can effectively antagonize the blood glucose reduction induced by exogenous GLP-1 after administration of 100 nmol/kg and 200 nmol/kg. On the contrary, P16289 antibody cannot affect the regulation of GLP-1 on blood glucose.

The regulatory role of P16288 on blood glucose can last for more than 8 days, indicating that P16288 has a very long half-life. In contrast, according to WO2021/231366A1, Avexitide (exendin-9) exhibits its effect of regulating blood glucose level for only a few hours due to its short half-life.

### EXAMPLE 6: A STUDY OF ANTIBODIES IN ORAL GLUCOSE TOLERANCE TEST (OGTT) IN HUMANIZED GLP-1R MICE (NO EXOGENOUS GLP-1 ADDED)

10-week-old male humanized GLP-1R mice (25-32 g) were assigned into 4 groups, with 5 mice in each group. The antibody treatment groups were subcutaneously injected with P16288 (200 nmol/kg), P16288 (400 nmol/kg) or P16289 (400 nmol/kg), respectively, and the vehicle group was subcutaneously injected with vehicle (PBS) once a week for 2 consecutive weeks. The first subcutaneous administration of antibody was on Day 0, and the second subcutaneous administration was on Day 7. OGTTs were performed on Day 1 and Day 8, and glucose solution (25%, w/v) was given to mice by oral administration at a dose of 13.9 mmol/kg. There was an 18-hour fasting period between the administration of the antibody and the oral administration of glucose solution. The blood glucose levels of the mice before (0 min) and 15 min, 30 min, 60 min, 90 min, 120 min and 180 min (or 240 min) after the administration of glucose were measured with blood glucose meter. The AUCs of the blood glucose-time curves of each mouse from 0 to 180 min (or 240 min) were calculated with GraphPad Prism 9. The blood glucose levels and the AUCs were shown in Mean±SEM and the differences among groups were compared. Statistical differences were analyzed with One-way ANOVA. As compared with the vehicle group, * stands for p<0.05; ** stands for p<0.01; *** stands for p<0.001; **** stands for p<0.0001; ns stands for no significance. The significance markers were not shown in the line chart since they would overlap and be difficult to distinguish.

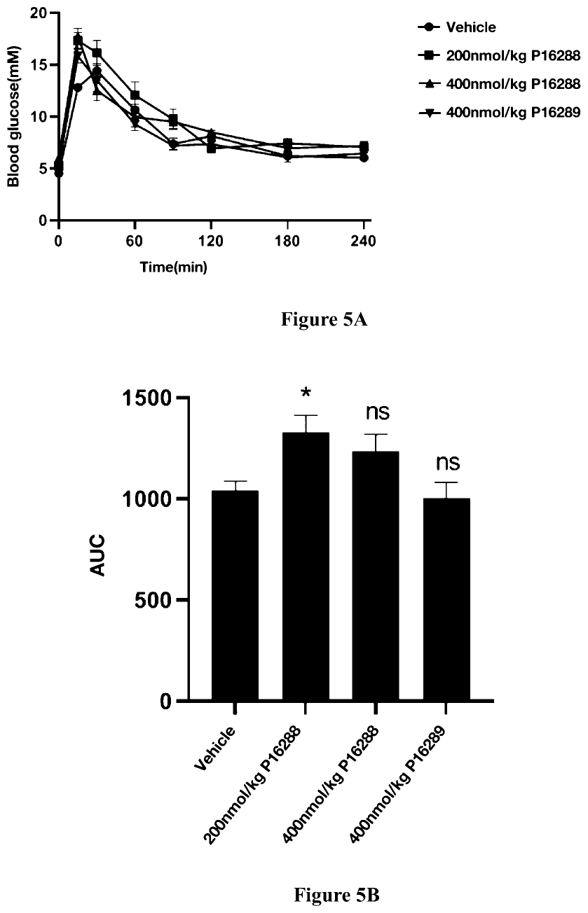

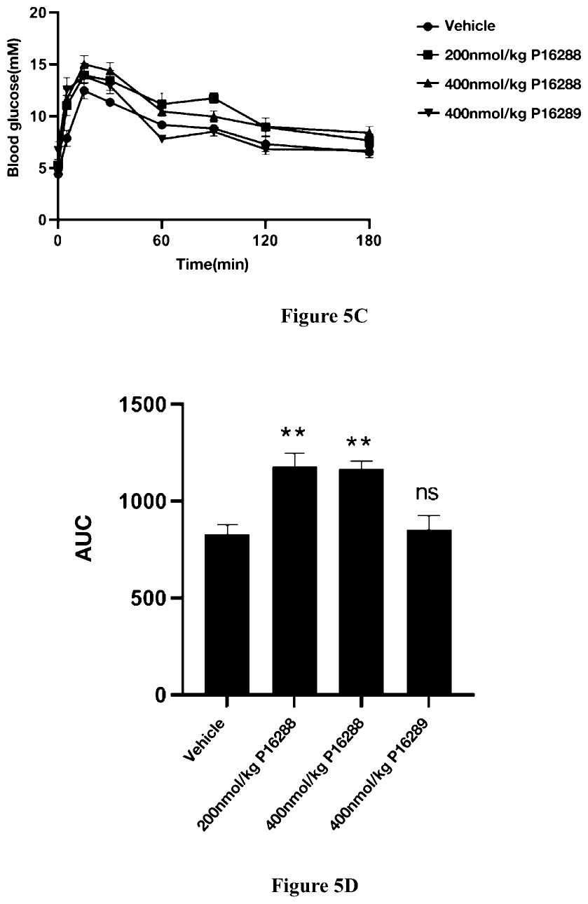

As shown in FIG. 5A-D, in the absence of exogenous GLP-1, the blood glucose-time curves first increase and then decrease after oral administration of glucose in humanized GLP-1R mice. In this process of blood glucose regulation, endogenous GLP-1 and insulin played a major role. After the mice were injected with P16288 and P16289. P16288 exhibited its effect of raising blood glucose level after both of the two administrations, while P16289 did not exhibit the effect of raising blood glucose level. This indicates that P16288 affects the regulation of blood glucose level of endogenous GLP-1 by antagonizing the activation of hGLP-1R by GLP-1.

### EXAMPLE 7: A STUDY OF THE ANTIBODY IN IPGTT IN C57BL/6 MICE (EXOGENOUS GLP-1 ADDED)

7-9-week-old male C57BL/6 mice (22-26 g) were assigned into 5 groups, with 5 mice in each group, and the mice in each group were treated according to the scheme shown in Table 6. The antibody treatment groups were given a single subcutaneous injection, and were fasted for 18 hours after the administration. After the fasting, exogenous GLP-1 and glucose solution were dosed to mice by intraperitoneal injection at 25.0 nmol/kg and 13.9 mmol/kg, respectively. The blood glucose levels of the mice before (0 min) and 15 min, 30 min. 60 min, 90 min, 120 min and 180 min after the administration of glucose were measured with blood glucose meter. The AUCs of the blood glucose-time curves of each mouse from 0 to 180 min were calculated with GraphPad Prism 9. The blood glucose levels and the AUCs were shown in Mean±SEM and the differences among groups were compared. Statistical differences were analyzed with One-way ANOVA. As compared with the group treated with GLP-1 alone. * stands for p<0.05; ** stands for p<0.01; *** stands for p<0.001; **** stands for p<0.0001; ns stands for no significance. The significance markers were not shown in the line chart since they would overlap and be difficult to distinguish.

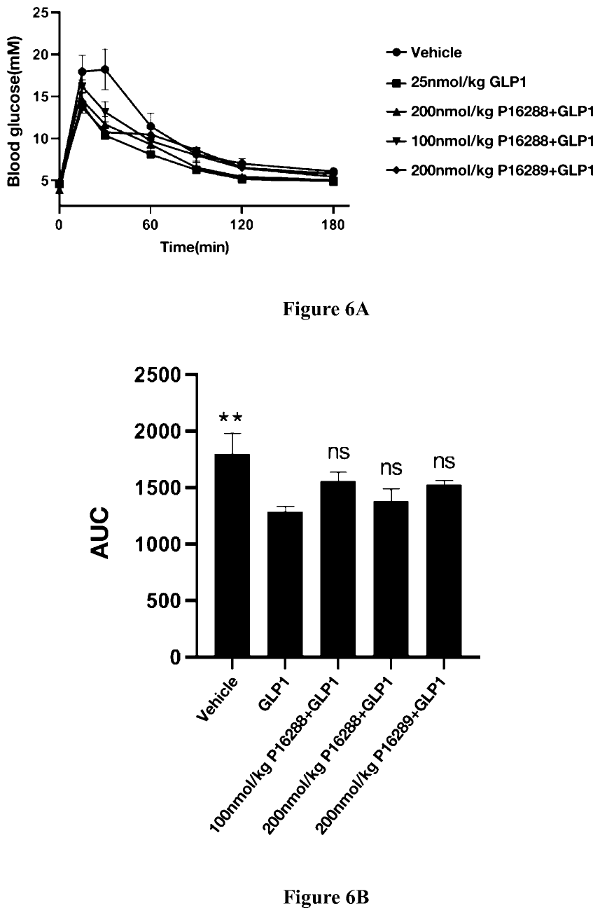

Since P16288 did not recognize mouse GLP-1R, the antibody should not exhibit its effect of regulating the receptor or blood glucose level in wildtype C57BL/6 mice, which further verified the specificity of the antibody activity. In this glucose tolerance study, GLP-1 could lower blood glucose levels, but P16288 did not exhibit the effect of antagonizing GLP-1 to raise blood glucose level as shown in FIG. 6A-B. This indicates that P16288 regulates blood glucose level through human GLP-1R specifically.

### EXAMPLE 8: A STUDY OF THE ANTIBODY IN OGTT IN C57BL/6 MICE (NO EXOGENOUS GLP-1 ADDED)

6-week-old male C57BL/6 mice (18-21 g) were assigned into 4 groups, with 5 mice in each group. Each group was given a single subcutaneous injection of vehicle (PBS), P16288 (200 nmol/kg), P16288 (100 nmol/kg) or P16289 (100 nmol/kg), and were fasted for 18 hours after administration. After the fasting, glucose solution was given to mice by oral administration at a dose of 13.9 mmol/kg. The blood glucose levels of the mice before (0 min) and 15 min. 30 min, 60 min, 90 min, 120 min, 180 min and 270 min after the administration of glucose were measured with blood glucose meter. The AUCs of the blood glucose-time curves of each mouse from 0 to 270 min were calculated with GraphPad Prism 9. The blood glucose levels and the AUCs were shown in Mean±SEM and the differences among groups were compared. Statistical differences were analyzed with One-way ANOVA. As compared with the vehicle group, * stands for p<0.05; ** stands for p<0.01; *** stands for p<0.001; stands for p<0.0001: ns stands for no significance. The significance markers were not shown in the line chart since they would overlap and be difficult to distinguish.

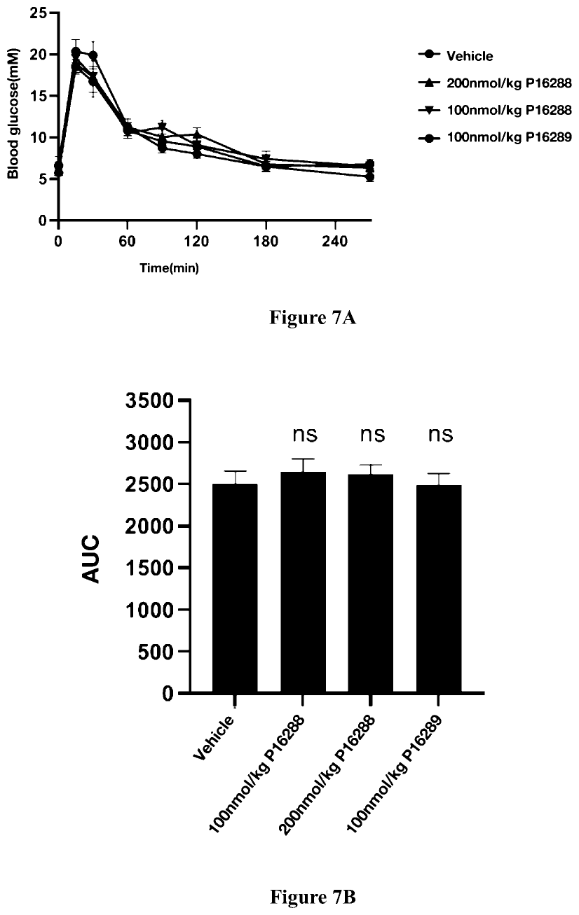

As shown in FIG. 7A-B, the results indicate that the blood glucose-time curves in OGTT of wildtype C57BL/6 mice are time-dependent. After the mice were injected with P16288, the blood glucose levels showed no statistical difference from the vehicle group. This also indicates that P16288 regulates blood glucose through human GLP-1R specifically.

### EXAMPLE 9: A STUDY ON THE PHARMACOKINETICS OF THE ANTIBODIES IN SD RATS

6-8-week-old male SD rats were given a single subcutaneous injection of P16288 or P16289 at a dose of 5 mg/kg, with 3 rats in each group. The whole blood of the rats at 5 minutes before administration (−5 min) and 2 h, 4 h, 24 h, 48 h, 72 h, 96 h, 120 h, 168 h, 192 h, 240 h, 264 h after administration were collected to prepare EDTA plasma. The concentration of antibodies in plasma was measured by ELISA. The detailed procedure of the measurement is as follow: The 96-well plate was coated with 1 μg/mL of AffiniPure Goat Anti-Human IgG antibody (Jackson lab, cat. no. 109-005-098) diluted in PBS and incubated overnight at 4° C. The coating solution was removed and the plate was washed with PBST (PBS+0.05% Tween). Then the plate was blocked with 1% BSA, wash with PBST, and added with the standard (P16288 or 16289) diluted in PBS+0.1% BSA+0.1% blank rat plasma at gradient concentrations of 1000, 200, 40, 8, 1.6, 0.32, 0.064, 0 nM, which was used for plotting a standard curve. At the same time, the plasma samples diluted 1000 times in PBS+0.1% BSA were added to other wells, and the plate was incubated at room temperature for 60 min. After washed with PBST, horseradish peroxidase-labeled Goat Anti-Human IgG, Fcγ fragment specific (Jackson lab, cat. no. 109-035-098) was added and the plate was incubated for 60 min. After the plate was washed with PBST, TMB substrate was added for coloration. The reaction was finally terminated with 0.5 M H₂SO₄, and the absorbance was measured with microplate reader at 450 nm. Drug concentration-time curves were plotted with GraphPad Prism 9, and the pharmacokinetic parameters (Cₘₐₓ, Tₘₐₓ, T_{1/2}, AUC and MRT) of the drugs were calculated in non-compartmental model. The results are shown in Table 7.

TABLE 7

| | P16288 | P16289 |
|---|---|---|
| Parameter | (mean) | (mean) |
| Cₘₐₓ (μg/mL) | 15.2 | 12.5 |
| Tₘₐₓ (h) | 42.0 | 56.0 |
| T_{1/2} (h) | 99.1 | 47.6 |
| AUC_{0-inf obs} (h*μg/mL) | 2435.1 | 1376.0 |
| MRT_{0-inf obs} (h) | 132.2 | 96.4 |

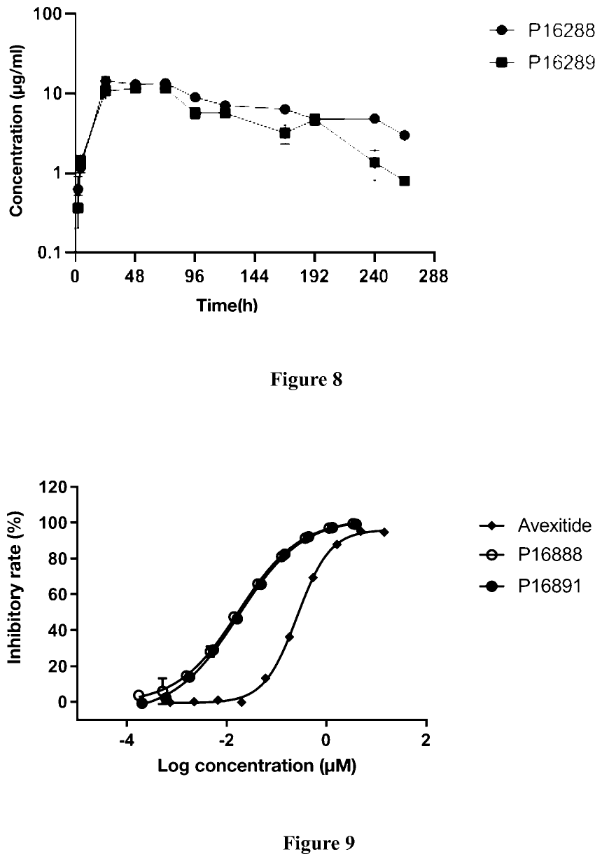

As shown in FIG. 8 and Table 7, P16288 in comparison with P16289 exhibits a longer half-life (T_{1/2}: 99.1 h) and a better exposure in the pharmacokinetic experiment of SD rats.

### EXAMPLE 10: A STUDY ON THE ANTAGONISM OF THE ANTIBODIES TO HUMAN OR MONKEY GLP-1R

### (1) A Cell-Based Assay on Antagonism Activity of the Antibodies to the Activation of Human Receptor by GLP-1

50 μL of hGLP-1R-CRE-Luciferase-HEK293 (6*10⁵ cells/mL) was added to a 96-well plate, followed by 50 μL P16888 of ten levels of concentrations in a 3-fold dilution gradient of 3425 nM, 1142 nM, 381 nM and so on, P16891 of ten levels of concentrations in a 3-fold dilution gradient of 3973 nM, 1324 nM, 441 nM and so on, Avexitide of ten levels of concentrations in a 3-fold dilution gradient of 14706 nM, 4902 nM, 1634 nM and so on, or equal volume of culture medium (minimum and maximum inhibition control groups). The plate was incubated at 37° C. in a 5% CO₂ incubator for 30 minutes, then added with GLP-1 to the minimal inhibition control group and antibody treatment groups to reach a final concentration of 2.5 ng/ml, and culture medium of equal volume to the maximal inhibition group, and further incubated overnight. 100 μL of Bright-Glo reagent (Promega Corporation) was added, and the plate was tapped gently to facilitate the mixing of the solution. Chemiluminescence value was read with microplate reader after three minutes. The maximum inhibition control group without GLP-1 treatment was defined as 100% inhibition rate, and the minimum inhibition control group treated with 2.5 ng/mL GLP-1 alone was defined as 0% inhibition rate. The data were analyzed by four-parameter curve fitting with GraphPad Prism 9, and IC₅₀ was calculated.

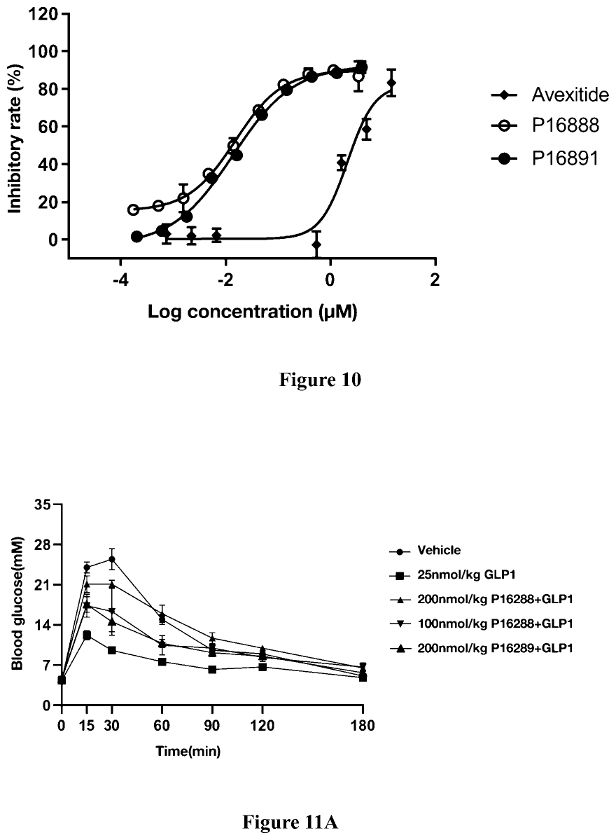

The results illustrated in FIG. 9 and Table 8 show that both P16888 and P16891 antagonize the activation of human GLP-1R by GLP-1, and the activities of P16888 and P16891 are higher than that of Avexitide.

TABLE 8

| | |
|---|---|
| P16888 | IC₅₀ = 17.4 nM |
| P16891 | IC₅₀ = 17.2 nM |
| Avexitide | IC₅₀ = 260.3 nM |

### (2) a Cell-Based Assay on Antagonism Activity of the Antibodies to the Activation of Monkey Receptor by GLP-1

50 μL of monkey GLP-1R (GenBank: JN033215.1)-CRE-Luciferase-HEK293 (6*10⁵ cells/mL) was added to a 96-well plate, followed by 50 μL P16888 of ten levels of concentrations in a 3-fold dilution gradient of 3425 nM, 1142 nM, 381 nM and so on. P16891 of ten levels of concentrations in a 3-fold dilution gradient of 3973 nM, 1324 nM, 441 nM and so on, Avexitide of ten levels of concentrations in a 3-fold dilution gradient of 14706 nM, 4902 nM. 1634 nM and so on, or equal volume of culture medium (minimum and maximum inhibition control groups). The plate was incubated at 37° C. in a 5% CO₂ incubator for 30 minutes, then added with GLP-1 to the minimal inhibition control group and antibody treatment groups to reach a final concentration of 2.5 ng/mL, and culture medium of equal volume to the maximal inhibition group, and further incubated overnight. 100 μL of Bright-Glo reagent (Promega Corporation) was added, and the plate was tapped gently to facilitate the mixing of the solution. Chemiluminescence value was read with microplate reader after three minutes. The maximum inhibition control group without GLP-1 treatment was defined as 100% inhibition rate, and the minimum inhibition control group treated with 2.5 ng/mL GLP-1 alone was defined as 0% inhibition rate. The data were analyzed by four-parameter curve fitting with GraphPad Prism 9, and IC₅₀ was calculated.

The results illustrated in FIG. 10 and Table 9 show that both P16888 and P16891 antagonize the activation of monkey GLP-1R by GLP-1, and the activities of P16888 and P16891 are higher than that of Avexitide.

TABLE 9

| | |
|---|---|
| P16888 | IC₅₀ = 15.3 nM |
| P16891 | IC₅₀ = 13.8 nM |
| Avexitide | IC₅₀ = 2118 nM |

### EXAMPLE 11: A STUDY OF ANTIBODY IN IPGTT IN HUMANIZED GLP-1R MICE (EXOGENOUS GLP-1 ADDED)

10-week-old female humanized GLP-1R mice (25-28 g) were assigned into 5 groups, with 5 mice in each group, and each group was treated according to the scheme in Table 10. The antibody treatment groups were subcutaneously injected with antibody, and the IPGTTs were performed on the next day of administration. The antibody was administered subcutaneously on Day 0, and the mice were fasted for 18 hours after the administration. After the fasting (Day 1), exogenous GLP-1 and glucose solution were dosed to mice by intraperitoneal injection at 25.0 nmol/kg and 13.9 mmol/kg, respectively. The blood glucose levels of the mice before (0 min) and 15 min, 30 min, 60 min, 90 min, 120 min and 180 min after the administration of glucose were measured with blood glucose meter. The AUCs of the blood glucose-time curves of each mouse were calculated with GraphPad Prism 9. The blood glucose levels and the AUCs were shown in Mean±SEM and the differences among groups were compared. Statistical differences were analyzed with One-way ANOVA. As compared with the GLP-1 individual treatment groups, * stands for p<0.05; ** stands for p<0.01; *** stands for p<0.001; ns stands for no significance.

TABLE 10

| | Dose of | Dose of | Dose of |
|---|---|---|---|
| Group | Antibody | GLP-1 | Glucose |
| control | none | none | 13.9 mmol/kg |
| 25 nmol/kg GLP-1 | none | 25.0 nmol/kg | 13.9 mmol/kg |
| 50 nmol/kg P16888 + | 50 nmol/kg | 25.0 nmol/kg | 13.9 mmol/kg |
| GLP-1 | P16888 | | |
| 100 nmol/kg P16888 + | 100 nmol/kg | 25.0 nmol/kg | 13.9 mmol/kg |
| GLP-1 | P16888 | | |
| 200 nmol/kg P16888 + | 200 nmol/kg | 25.0 nmol/kg | 13.9 mmol/kg |
| GLP-1 | P16888 | | |

As shown in FIGS. 11A and 11B, P16888 exhibits a dose-dependent effect of raising blood glucose. 200 nmol/kg of P16888 antibody can raise the blood glucose AUC to that of the control group without GLP-1 treatment after a single administration.

### EXAMPLE 12: A STUDY ON THE DURATION OF THE EFFICACY OF P16888 ANTIBODY IN HUMANIZED GLP-1R MICE (EXOGENOUS GLP-1 ADDED)

A method similar to that of Example 11 was adopted. 10-week-old female humanized GLP-1R mice (25-28 g) with 5 mice in each group were selected and the antibody treatment group was given a single subcutaneous injection of P16888. The IPGTTs were performed on Day 2, Day 9, Day 16 and Day 23 after the administration. The first subcutaneous administration of the antibody was recorded as Day 0. Mice were fasted for 18 hours after administration or before IPGTTs. After the fasting (Day 1, Day 8, Day 15, Day 22), exogenous GLP-1 (human, R&D Systems) and glucose solution were dosed to mice by intraperitoneal injection at 25.0 nmol/kg and 13.9 mmol/kg, respectively. The blood glucose levels of the mice before (0 min) and 30 min, 60 min. 90 min, 120 min and 180 min after the administration of glucose were measured with blood glucose meter. The AUCs of the blood glucose-time curves of each mouse were calculated with GraphPad Prism 9. The blood glucose levels and the AUCs were shown in Mean±SEM and the differences among groups were compared.

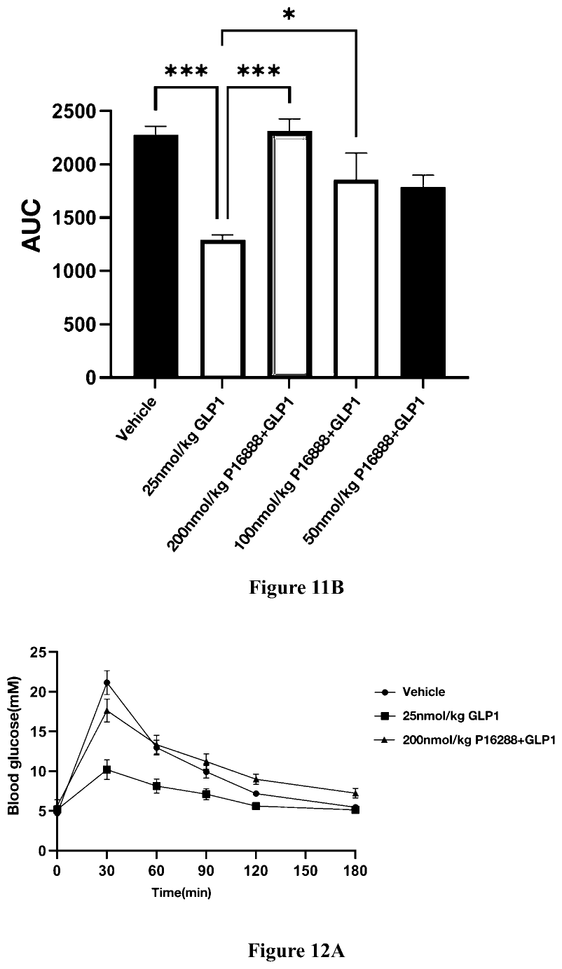

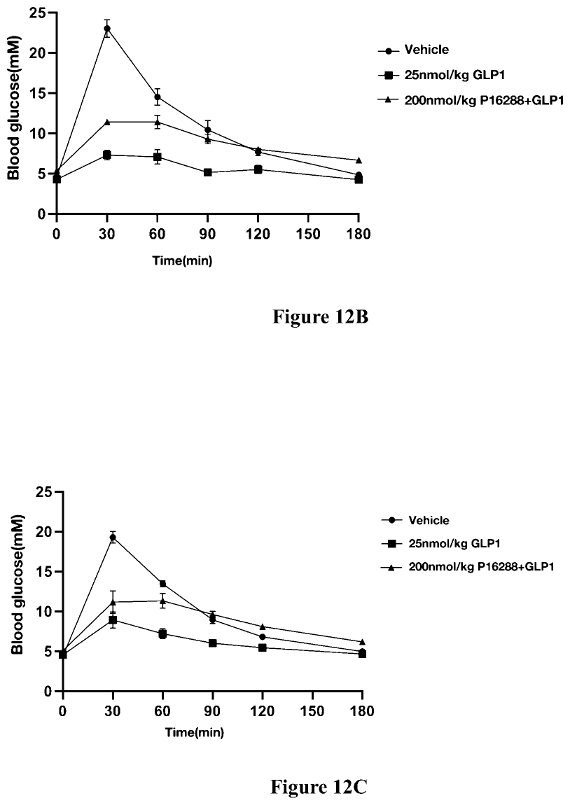

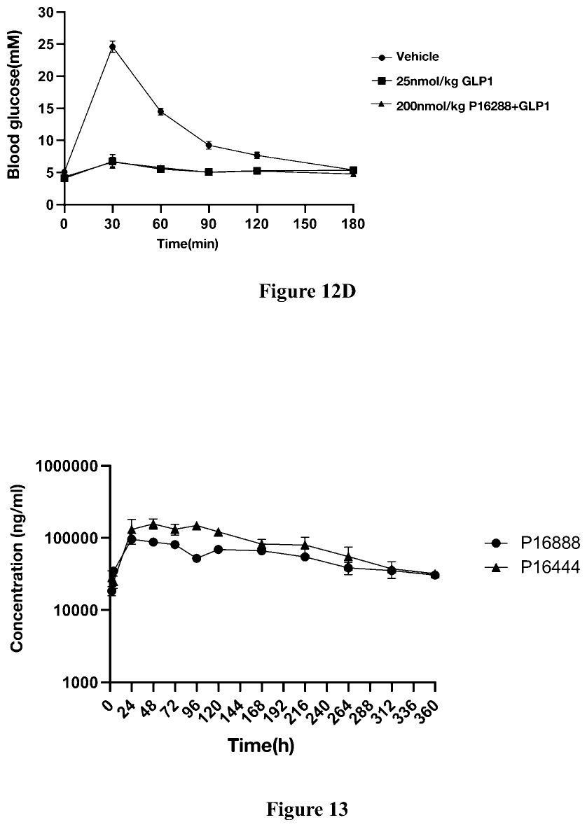

The results are shown in FIG. 12A-D. In the IPGTT pharmacodynamic experiment of humanized GLP-1R mice, P16888 exhibited a long-lasting effect of raising blood glucose. After a single subcutaneous injection of the antibody, the effect of raising blood glucose lasted for more than 15 days after the administration (compared with the GLP-1 treatment group, the p values of the AUCs of the blood glucose-time curves of each treatment group at the corresponding days were all less than 0.001), indicating that the antibody has a long-lasting effect.

### EXAMPLE 13: A STUDY ON THE PHARMACOKINETICS OF ANTIBODIES IN HUMANIZED GLP-1R MICE

P16444 antibody was used as control to P16888 antibody disclosed herein. The sequence of P16444 can be found in the name mAb36986 in WO2021/231366A1.

10-week-old female humanized GLP-1R mice (25-28 g) were given a single subcutaneous injection of 5 mg/kg (34 nmol/kg) of the antibody P16888 or P16444 (n=4). The whole blood samples were collected before administration (−5 min) and 2 h, 4 h, 24 h, 48 h, 72 h, 96 h, 120 h, 168 h, 216 h, 264 h, 312 h and 360 h after administration to prepare plasma samples. The antibody concentrations in plasma were measured by ELISA. Drug concentration-time curves were plotted with GraphPad Prism 9, and the pharmacokinetic parameters (Cₘₐₓ, Tₘₐₓ, T_{1/2}, AUC and MRT) were calculated in non-compartmental model.

TABLE 11

| | P16888 | P16444 |
|---|---|---|
| Parameter | (mean) | (mean) |
| Cₘₐₓ (ng/mL) | 96090.5 | 15757.2 |
| Tₘₐₓ (h) | 24.0 | 48.0 |
| T_{1/2} (h) | 289.4 | 121.9 |
| AUC_{0-inf obs} (h*ng/mL) | 33641009.6 | 36077118.7 |
| MRT_{0-inf obs} (h) | 213.4 | 163 |

The results illustrated in FIG. 13 and Table 11 show that the half-life of P16888 is 289.4 h, which is much longer than that of Avexitide: about 30 min (Gasbjerg L S, et al. Diabetes, Obesity & Metabolism. 2021 November; 23 (11): 2419-2436.), and also longer than that of the control antibody P16444.

### EXAMPLE 14: A STUDY ON THE PHARMACOKINETICS OF ANTIBODIES IN CYNOMOLGUS MONKEYS

3-4-year-old male cynomolgus monkeys were given a single subcutaneous injection of 1 mg/kg antibody (P16888 or P16891) (n=2-3). The whole blood samples were collected before administration (−5 min) and 0.5 h, 4 h, 8 h, 24 h, 48 h, 72 h. 96 h, 120 h. 168 h, 240 h, 336 h, 504 h, 672 h, 840 h, 1008 h and 1344 h after administration to prepare plasma samples. The antibody concentrations in plasma were measured by ELISA. Drug concentration-time curves were plotted with GraphPad Prism 9, and the pharmacokinetic parameters (Cₘₐₓ, Tₘₐₓ, T_{1/2}, AUC and MRT) were calculated in non-compartmental model.

TABLE 12

| | P16888 | P16891 |
|---|---|---|
| Parameter | (mean) | (mean) |
| Cₘₐₓ (ng/mL) | 17395.40 | 15211.90 |
| Tₘₐₓ (h) | 84.0 | 96.0 |
| T_{1/2} (h) | 330.20 | 529.15 |
| AUC_{0-inf obs} (h*ng/mL) | 10934659.89 | 11717313.32 |
| MRT_{0-inf obs} (h) | 465.79 | 531.61 |

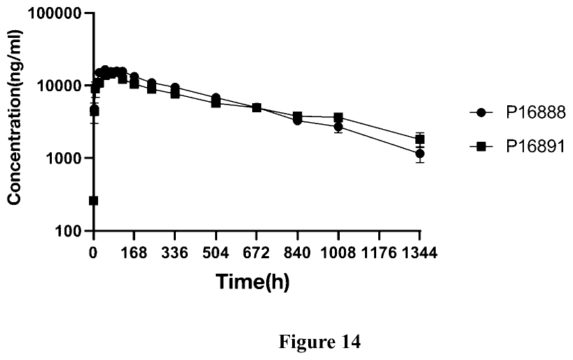

The results illustrated in FIG. 14 and Table 12 show that the half-life of P16888 is 330 h and that of P16891 is even longer, up to 529 h.

### EXAMPLE 15: A STUDY ON THE THERMAL STABILITY OF THE ANTIBODY

1 mg/ml of P16888 antibody was prepared in PBS of pH 5.5 or PH 7.4, and was incubated at 40° C. Samples were taken on Day 0, Day 10, Day 20 and Day 30, and each sample was subject to test for cell activity and SEC-HPLC. The activity of the cells was measured by hGLP-1R-CRE-Luciferase-HEK293 reporter gene assay as illustrated in Example 3.

TABLE 13

| | Average IC₅₀, nM | | | | Relative IC₅₀ compared to Day 0 | | | | SEC, % Content of Main Peak | | | |
|---|---|---|---|---|---|---|---|---|---|---|---|---|
| Condition | Day 0 | Day 10 | Day 20 | Day 30 | Day 0 | Day 10 | Day 20 | Day 30 | Day 0 | Day 10 | Day 20 | Day 30 |
| PBS, PH 5.5 | 6.8 | 5.9 | 6.7 | 7.4 | 1 | 0.8 | 1.0 | 1.0 | 96.32 | 97.10 | 97.34 | 97.44 |
| PBS, PH 7.4 | 19.7 | 20.6 | 18.4 | 18.7 | 1 | 1.0 | 0.9 | 1.0 | 95.96 | 96.69 | 96.48 | 96.35 |

As shown in Table 13, P16888 antibody has good stability at both pH 5.5, 40° C. and PH 7.4, 40° C.

1-23. (canceled)

24. A monoclonal antibody or antigen-binding fragment thereof, comprising:
a heavy chain complementarity-determining region 1 (HCDR1) of amino acid sequence of SEQ ID NO: 2, or having one or more conservative amino acid substitutions as compared to SEQ ID NO: 2;
a heavy chain complementarity-determining region 2 (HCDR2) of amino acid sequence of SEQ ID NO: 3, or having one or more conservative amino acid substitutions as compared to SEQ ID NO: 3;
a heavy chain complementarity-determining region 3 (HCDR3) of amino acid sequence of SEQ ID NO: 4 or SEQ ID NO: 22, or having one or more conservative amino acid substitutions as compared to SEQ ID NO: 4;
a light chain complementarity-determining region 1 (LCDR1) of amino acid sequence of SEQ ID NO: 6, or having one or more conservative amino acid substitutions as compared to SEQ ID NO: 6;
a light chain complementarity-determining region 2 (LCDR2) of amino acid sequence of SEQ ID NO: 7, or having one or more conservative amino acid substitutions as compared to SEQ ID NO: 7; and
a light chain complementarity-determining region 3 (LCDR3) of amino acid sequence of SEQ ID NO: 8 or SEQ ID NO: 24, or having one or more conservative amino acid substitutions as compared to SEQ ID NO: 8, wherein:
if there is a conservative amino acid substitution in HCDR1, HCDR2, and/or HCDR 3, then the sum of the numbers of the substitution(s) in HCDR1, HCDR2, and HCDR 3 is 1, 2, or 3; and
if there is a conservative amino acid substitution in LCDR1, LCDR2, and/or LCDR 3, then the sum of the numbers of the substitution(s) in LCDR1, LCDR2, and LCDR 3 is 1, 2, or 3.

25. The monoclonal antibody or antigen-binding fragment thereof according to claim 24, comprising:
the HCDR1 of amino acid sequence of SEQ ID NO: 2;
the HCDR2 of amino acid sequence of SEQ ID NO: 3;
the HCDR3 of amino acid sequence of SEQ ID NO: 4 or SEQ ID NO: 22, or having one conservative amino acid substitution as compared to SEQ ID NO: 4;
the LCDR 1 of amino acid sequence of SEQ ID NO: 6;
the LCDR2 of amino acid sequence of SEQ ID NO: 7; and
the LCDR3 of amino acid sequence of SEQ ID NO: 8 or SEQ ID NO: 24, or having one conservative amino acid substitution as compared to SEQ ID NO: 8.

26. The monoclonal antibody or antigen-binding fragment thereof according to claim 24, comprising:
a heavy chain variable region (HCVR) of an amino acid sequence of SEQ ID NO: 1 or 21, or having at least 90% identity to the amino acid sequence of SEQ ID NO: 1 or 21; and/or
a light chain variable region (LCVR) of an amino acid sequence of SEQ ID NO: 5 or 23, or having at least 90% identity to the amino acid sequence of SEQ ID NO: 5 or 23.

27. The monoclonal antibody or antigen-binding fragment thereof according to claim 24, comprising:
a heavy chain of the amino acid sequence of SEQ ID NO: 9, SEQ ID NO: 25, or SEQ ID NO: 27; and
a light chain of the amino acid sequence of SEQ ID NO: 10 or SEQ ID NO: 26.

28. The monoclonal antibody or antigen-binding fragment thereof according to claim 24, comprising:
a heavy chain constant region selected from the heavy chain constant region of human IgG1, IgG2, IgG3, IgG4 or conventional variants thereof; and
a light chain constant region selected from the light chain constant region of human k chain, A chain or conventional variants thereof,
wherein the monoclonal antibody or antigen-binding fragment thereof is a full-length IgG antibody.

29. The monoclonal antibody or antigen-binding fragment thereof according to claim 24, wherein the monoclonal antibody or antigen-binding fragment thereof is a fully human antibody.

30. The monoclonal antibody or antigen-binding fragment thereof according to claim 24 capable of specifically binding to human glucagon-like peptide 1 receptor (GLP-1R) and antagonizing the activation of human GLP-1R by GLP-1.

31. A monoclonal antibody or antigen-binding fragment thereof which competes for binding GLP-1R with the monoclonal antibody or antigen-binding fragment thereof of claim 24.

32. A nucleic acid encoding the monoclonal antibody or antigen-binding fragment thereof according to claim 24.

33. A vector comprising a nucleic acid encoding the monoclonal antibody or antigen-binding fragment thereof according to claim 24.

34. A cell, comprising:
the monoclonal antibody or antigen-binding fragment thereof according to claim 24;
a nucleic acid encoding the monoclonal antibody or antigen-binding fragment thereof according to claim 24; and/or
a vector comprising a nucleic acid encoding the monoclonal antibody or antigen-binding fragment thereof according to claim 24.

35. A pharmaceutical composition, comprising:
the monoclonal antibody or antigen-binding fragment thereof of claim 24; and
a pharmaceutically acceptable carrier.

36. A kit, comprising:
the monoclonal antibody or antigen-binding fragment thereof of claim 24;
a nucleic acid encoding the monoclonal antibody or antigen-binding fragment thereof according to claim 24;
a vector comprising a nucleic acid encoding the monoclonal antibody or antigen-binding fragment thereof according to claim 24; and/or
a cell, comprising:
the monoclonal antibody or antigen-binding fragment thereof according to claim 24;
a nucleic acid encoding the monoclonal antibody or antigen-binding fragment thereof according to claim 24; and/or
a vector comprising a nucleic acid encoding the monoclonal antibody or antigen-binding fragment thereof according to claim 24.

37. A method of preparing the monoclonal antibody or antigen-binding fragment thereof according to claim 24, comprising:
introducing a polynucleotide sequence encoding the monoclonal antibody or antigen-binding fragment thereof according to claim 24 into an expression vector;
transforming, transfecting or transducing the expression vector into an expression system to obtain an expression product; and
separating and purifying the expression product.

38. A method of treating, preventing or ameliorating
a symptom or condition of a GLP-1R-associated disease or disorder, or
a symptom or condition of a hypoglycemia-associated disease or disorder,
comprising:
administering a therapeutically effective amount of the monoclonal antibody or antigen-binding fragment thereof of claim 24 to a subject.

39. The method according to claim 38, wherein said GLP-1R-associated disease or disorder is GLP-1R-hyperactivity-associated disease or disorder.

40. The method according to claim 38, wherein said GLP-1R-associated disease or disorder is hypoglycemia.

41. The method according to claim 38, wherein said hypoglycemia-associated disease or disorder is neurological damage or developmental complications caused by hypoglycemia or congenital hyperinsulinism (HI).

42. The method according to claim 38, wherein the symptom or condition of said hypoglycemia-associated disease or disorder is hypoglycemia, cerebral damage, developmental delay, feeding disorder, learning disability, and/or epilepsy.

43. The method according to claim 38, wherein said monoclonal antibody or antigen-binding fragment thereof is administered
subcutaneously, intraperitoneally, intravenously, intramuscularly or topically; and/or
in combination with a second therapeutic agent.


SEQUENCE LISTING (27 sequences)

SEQ ID NO: 1 - protein, 121 aa, synthetic construct
QVQLVQSGAEMKKPGASVKVSCKTSGYRFSTYVISWVRQTPGQGLEWMGWISPYTGDIDYAQKFQGRVTITADESTSTAYMELSRLRSDDTAVYYCARERDGYNFPGMDVWGQGTTVTVSS

SEQ ID NO: 2 - protein, 5 aa, synthetic construct
TYVIS

SEQ ID NO: 3 - protein, 17 aa, synthetic construct
WISPYTGDIDYAQKFQG

SEQ ID NO: 4 - protein, 12 aa, synthetic construct
ERDGYNFPGMDV

SEQ ID NO: 5 - protein, 107 aa, synthetic construct
DIQLTQSPSFLSASVGDRVTITCRASQGISSYLAWYQQKPGRAPKLLIYSASTLQRGVPSRFSGSGSGTDFTLTISSLQPEDYATYYCQQADSFPPSFGPGTKVDIK

SEQ ID NO: 6 - protein, 11 aa, synthetic construct
RASQGISSYLA

SEQ ID NO: 7 - protein, 7 aa, synthetic construct
SASTLQR

SEQ ID NO: 8 - protein, 9 aa, synthetic construct
QQADSFPPS

SEQ ID NO: 9 - protein, 448 aa, synthetic construct
QVQLVQSGAEMKKPGASVKVSCKTSGYRFSTYVISWVRQTPGQGLEWMGWISPYTGDIDYAQKFQGRVTITADESTSTAYMELSRLRSDDTAVYYCARERDGYNFPGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQLTQSPSFLSASVGDRVTITCRASQGISSYLAWYQQKPGRAPKLLIYSASTLQRGVPSRFSGSGSGTDFTLTISSLQPEDYATYYCQQADSFPPSFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 126 aa, synthetic construct
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCAREGYSSSWTYYYYYYMDVWGKGTMVTVSS

SEQ ID NO: 12 - protein, 10 aa, synthetic construct
GFTFSSYAMH

SEQ ID NO: 13 - protein, 17 aa, synthetic construct
VISYDGSNKYYADSVKG

SEQ ID NO: 14 - protein, 19 aa, synthetic construct
AREGYSSSWTYYYYYYMDV

SEQ ID NO: 15 - protein, 107 aa, synthetic construct
DIQLTQSPSSLSASVGDRVTITCRASQGVSRALAWYQQKPGKAPKLLIYDASYLEDGVPSRFSGSGSGTDFTLTISSLQPEDFATYYCQQSYSTPYTFGQGTKLEIK

SEQ ID NO: 16 - protein, 11 aa, synthetic construct
RASQGVSRALA

SEQ ID NO: 17 - protein, 7 aa, synthetic construct
DASYLED

SEQ ID NO: 18 - protein, 9 aa, synthetic construct
QQSYSTPYT

SEQ ID NO: 19 - protein, 453 aa, synthetic construct
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCAREGYSSSWTYYYYYYMDVWGKGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, synthetic construct
DIQLTQSPSSLSASVGDRVTITCRASQGVSRALAWYQQKPGKAPKLLIYDASYLEDGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 121 aa, synthetic construct
QVQLVQSGAEMKKPGASVKVSCKTSGYRFSTYVISWVRQTPGQGLEWMGWISPYTGDIDYAQKFQGRVTITADESTSTAYMELSRLRSDDTAVYYCAREREGYNFPGMDVWGQGTTVTVSS

SEQ ID NO: 22 - protein, 12 aa, synthetic construct
EREGYNFPGMDV

SEQ ID NO: 23 - protein, 107 aa, synthetic construct
DIQLTQSPSFLSASVGDRVTITCRASQGISSYLAWYQQKPGRAPKLLIYSASTLQRGVPSRFSGSGSGTDFTLTISSLQPEDYATYYCQQAESFPPSFGPGTKVDIK

SEQ ID NO: 24 - protein, 9 aa, synthetic construct
QQAESFPPS

SEQ ID NO: 25 - protein, 448 aa, synthetic construct
QVQLVQSGAEMKKPGASVKVSCKTSGYRFSTYVISWVRQTPGQGLEWMGWISPYTGDIDYAQKFQGRVTITADESTSTAYMELSRLRSDDTAVYYCAREREGYNFPGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 214 aa, synthetic construct
DIQLTQSPSFLSASVGDRVTITCRASQGISSYLAWYQQKPGRAPKLLIYSASTLQRGVPSRFSGSGSGTDFTLTISSLQPEDYATYYCQQAESFPPSFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 448 aa, synthetic construct
QVQLVQSGAEMKKPGASVKVSCKTSGYRFSTYVISWVRQTPGQGLEWMGWISPYTGDIDYAQKFQGRVTITADESTSTAYMELSRLRSDDTAVYYCAREREGYNFPGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK